(19) United States
(12) Patent Application Publication
(10) Pub. No.: US 2026/0028319 A1
(43) Pub. Date: Jan. 29, 2026

(54) INTERMEDIATE SALT FOR THE PREPARATION OF BIS(FLUOROALKYL) BENZODIAZEPINONE COMPOUNDS

(71) Applicant: Immunome, Inc., Bothell, WA (US)

(72) Inventors: Christopher M. Cimarusti, Clementon, NJ (US); Ronen Ben David, Netanya (IL); David Caron, Even Yehuda (IL)

(21) Appl. No.: 19/143,803

(22) PCT Filed: Dec. 27, 2023
(86) PCT No.: PCT/US2023/085951

§ 371 Date: Jun. 26, 2025

Related U.S. Application Data
(60) Provisional application No. 63/435,592, filed on Dec. 28, 2022.

(51) Int. Cl. C07D 243/26; C07C 67/317; C07C 69/63; C07C 209/68; C07C 211/62

(52) U.S. Cl. CPC C07D 243/26; C07C 67/317; C07C 69/63; C07C 209/68; C07C 211/62; C07C 2601/14

(57) ABSTRACT

The present invention provides salts represented by the structure of Formula (I): wherein Q is an ammonium cation; and processes comprising the use of the salts in the preparation of benzodiazepinone compounds, especially bis(fluoroalkyl) derivatives thereof.

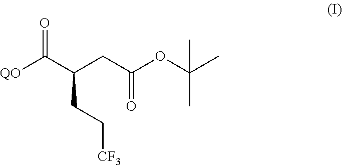

### FIELD OF THE INVENTION

The present invention provides salts represented by the structure of Formula (I):

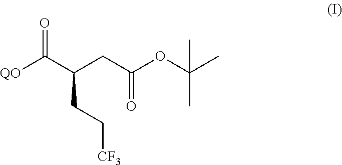

wherein
Q is an ammonium cation; and
processes comprising use of the salts in the preparation of benzodiazepinone compounds, especially bis(fluoroalkyl) derivatives thereof.

### BACKGROUND OF THE INVENTION

Bis(fluoroalkyl) benzodiazepinone compounds are useful as pharmaceutically active ingredients in the pharmaceutical and fine chemical industries. For example, the gamma-secretase inhibitors (GSIs):
(2R,3S)—N-((3S)-1-Methyl-2-oxo-5-phenyl-2,3-dihydro-1H-1,4-benzodiazepin-3-yl)-2,3-bis(3,3,3-trifluoropropyl)succinimide:

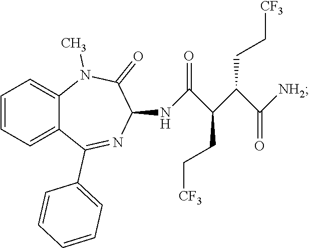 [SMILES: CN1C(=O)[C@@H](NC(=O)[C@H](CCC(F)(F)F)[C@H](CCC(F)(F)F)C(N)=O)N=C(c2ccccc2)c2ccccc21]

and
(2R,3S)—N-[(3S)-5-(3-Fluorophenyl)-9-methyl-2-oxo-2,3-dihydro-1H-1,4-benzodiazepin-3-yl]-2,3-bis(3,3,3-trifluoropropyl)succinimide:

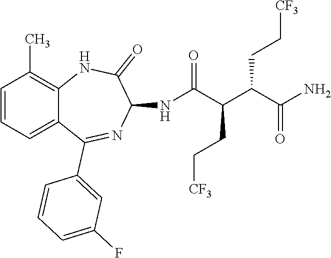 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

are showing promising results in current clinical trials for the treatment of various cancers, particularly cancers with defects in Notch regulation (see U.S. Pat. Nos. 9,273,014 and 8,629,136, incorporated herein by reference).

Within the preparation of (2R,3S)—N-((3S)-1-Methyl-2-oxo-5-phenyl-2,3-dihydro-1H-1,4-benzodiazepin-3-yl)-2,3-bis(3,3,3-trifluoropropyl)succinimide and (2R,3S)—N-[(3S)-5-(3-Fluorophenyl)-9-methyl-2-oxo-2,3-dihydro-1H-1,4-benzodiazepin-3-yl]-2,3-bis(3,3,3-trifluoropropyl)succinimide), the free acid (2R)-2-(2-tert-Butoxy-2-oxoethyl)-5,5,5-trifluoropentanoic acid is formed and used (Id.):

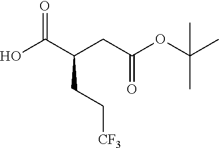 [SMILES: CC(C)(C)OC(=O)C[C@@H](CCC(F)(F)F)C(=O)O]

It was found that the free acid compound is oily and unstable.

Stability is a key consideration when selecting a synthetic intermediate in a process of drug preparation, especially when the process involves several synthetic stages. Each intermediate should be pure and stable, in order to limit impurities/degradation products in further synthetic stages, in which impurities may interfere with the chemical reactions of the synthesis, with the purity of the final drug substance as well as of the final drug product.

Stability is also beneficial in an intermediate synthetic product so that different synthetic stages may take place in different locations and/or at different times. Thus, prolonged stability, especially at room temperature or higher temperatures will ease the logistical requirements for synthesis, handling and transportation of the intermediate.

Accordingly, there is an unmet need to provide stable forms of (2R)-2-(2-tert-Butoxy-2-oxoethyl)-5,5,5-trifluoropentanoic acid to utilize in reliable and efficient synthetic processes of making (2R,3S)—N-((3S)-1-Methyl-2-oxo-5-phenyl-2,3-dihydro-1H-1,4-benzodiazepin-3-yl)-2,3-bis(3,3,3-trifluoropropyl)succinimide and (2R,3S)—N-[(3S)-5-(3-Fluorophenyl)-9-methyl-2-oxo-2,3-dihydro-1H-1,4-benzodiazepin-3-yl]-2,3-bis(3,3,3-trifluoropropyl)succinimide.

### SUMMARY OF THE INVENTION

In one embodiment, the present invention provides a salt represented by the structure of Formula (I):

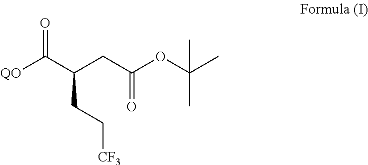

wherein Q is an ammonium cation.

In a further embodiment, the present invention provides a method of preparing compound (3a) or (3b), comprising steps A-B:
Step A:

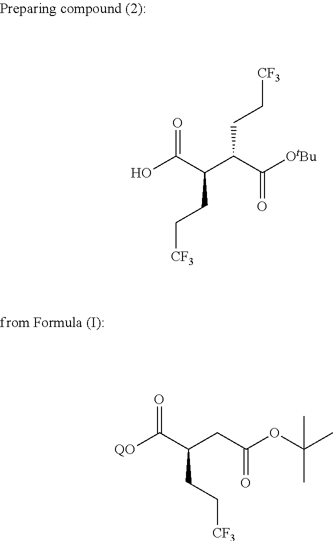

wherein
Q is an ammonium cation; and
Step B:

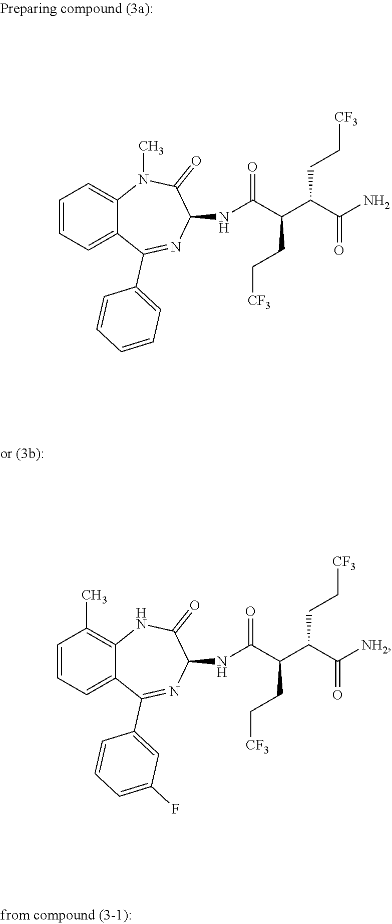 [SMILES: CN1C(=O)[C@@H](NC(=O)[C@H](CCC(F)(F)F)[C@H](CCC(F)(F)F)C(N)=O)N=C(c2ccccc2)c2ccccc21 ; Cc1cccc2c1NC(=O)[C@@H](NC(=O)[C@H](CCC(F)(F)F)[C@H](CCC(F)(F)F)C(N)=O)N=C2c1cccc(F)c1]

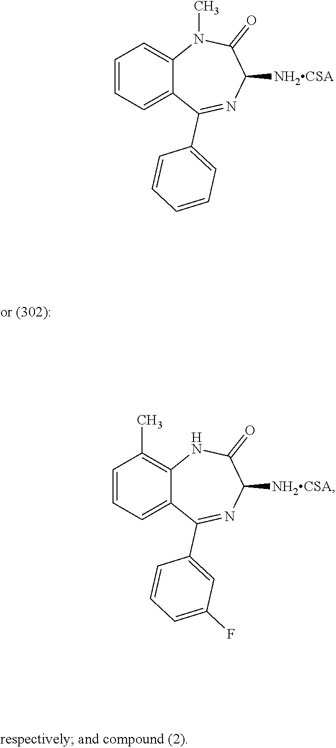

In some embodiments, Q of Formula (I) is a cyclohexyl ammonium cation.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Formula (I)

In one embodiment, the present invention provides salts represented by the structure of Formula (I):

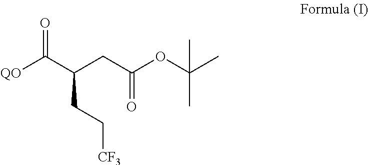

wherein
Q is an ammonium cation. In another embodiment, the ammonium cation is a primary, secondary or tertiary ammonium cation. In another embodiment, the primary ammonium cation comprises (as a substituent to the nitrogen) a cycloalkyl moiety. In another embodiment, non-limiting examples of the primary ammonium cation are selected from the group consisting of cyclohexyl, cyclopentyl, cycloheptyl and cyclooctyl ammonium cation. In another embodiment, non-limiting examples of the secondary ammonium cation are selected from the group consisting of di-cyclohexyl, di-cyclopentyl, di-cycloheptyl and di-cyclooctyl ammonium cation. In another embodiment, non-limiting examples of the tertiary ammonium cation are selected from the group consisting of di-isopropyl ethyl, diethyl-isopropyl and N-methylmorpholine ammonium cation. In another embodiment, Q is a cyclohexylammonium cation, and the compound is (1):

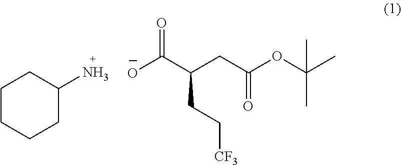

Each possibility represents a separate embodiment of the invention.

In one embodiment, the salts of Formula (I) are stable for 2-52 weeks at either room temperature, 2-8° C. or 40° C./75% RH (relative humidity). In one other embodiment, the salts of Formula (I) are stable for 2-30 weeks at either room temperature, 2-8° C. or 40° C./75% RH (relative humidity). In another embodiment, the salts are stable for 2-24 weeks. In another embodiment, the salts are stable for 2-20 weeks. In another embodiment, the salts are stable for 2-16 weeks. In another embodiment, the salts are stable for 2-8 weeks. In another embodiment, the salts are stable for 2-6 weeks. In another embodiment, the salts are stable for 2-4 weeks. In another embodiment, the salts are stable for 4-30 weeks. In another embodiment, the salts are stable for 4-24 weeks. In another embodiment, the salts are stable for 4-20 weeks. In another embodiment, the salts are stable for 4-16 weeks. In another embodiment, the salts are stable for 4-8 weeks. In another embodiment, the salts are stable for 4-6 weeks. In another embodiment, the salts are stable for 6-30 weeks. In another embodiment, the salts are stable for 6-24 weeks. In another embodiment, the salts are stable for 6-20 weeks. In another embodiment, the salts are stable for 6-16 weeks. In another embodiment, the salts are stable for 6-8 weeks. In another embodiment, the salts are stable for 8-30 weeks. In another embodiment, the salts are stable for 8-24 weeks. In another embodiment, the salts are stable for 8-20 weeks. In another embodiment, the salts are stable for 8-16 weeks. In another embodiment, the salts are stable for 16-30 weeks. In another embodiment, the salts are stable for 16-24 weeks. In another embodiment, the salts are stable for 16-20 weeks. In another embodiment, the salts are stable for 20-30 weeks. In another embodiment, the salts are stable for 20-24 weeks. In another embodiment, the salts are stable for 24-30 weeks. In another embodiment, the salts are stable for 24 weeks Each possibility represents a separate embodiment of the invention.

In one embodiment, the salts of Formula (I) have an initial purity (i.e. purity measured when prepared and isolated) of between 95 and 99.99%. In another embodiment, the purity is between 95-97%. In another embodiment, the purity is between 95-98%. In another embodiment, the purity is between 95-99%. In another embodiment, the purity is between 95-99.5%. In another embodiment, the purity is between 95-99.9%. In another embodiment, the purity is between 97-98%. In another embodiment, the purity is between 97-99%. In another embodiment, the purity is between 97-99.5%. In another embodiment, the purity is between 97-99.9%. In another embodiment, the purity is between 98-99%. In another embodiment, the purity is between 98-99.5%. In another embodiment, the purity is between 98-99.9%. In another embodiment, the purity is between 99-99.5%. In another embodiment, the purity is between 99-99.9%. In another embodiment, the purity is between 99.5-99.9%. In some embodiments, the purity is measured via HPLC, GC, GC-MS or NMR. Each possibility represents a separate embodiment of the invention.

In one embodiment, the salts of Formula (I) have an initial assay value of between 94 and 99.99%. In another embodiment, the assay is between 94-97%. In another embodiment, the assay is between 94-99.99%. In another embodiment, the assay is between 94-98%. In another embodiment, the assay is between 94-99%. In another embodiment, the assay is between 94-99.5%. In another embodiment, the assay is between 95-99.9%. In another embodiment, the assay is between 97-98%. In another embodiment, the assay is between 97-99%. In another embodiment, the assay is between 97-99.5%. In another embodiment, the assay is between 97-99.9%. In another embodiment, the assay is between 98-99%. In another embodiment, the assay is between 98-99.5%. In another embodiment, the assay is between 98-99.9%. In another embodiment, the assay is between 99-99.5%. In another embodiment, the assay is between 99-99.9%. In another embodiment, the assay is between 99.5-99.9%. In some embodiments, the assay is measured via HPLC, GC, GC-MS or NMR. Each possibility represents a separate embodiment of the invention. In some embodiments, the salt of Formula (I) is in a powder state. In one embodiment, the cyclohexylamine salt (CHA) (i.e., wherein Q is a cyclohexylammonium cation), i.e. compound (1) is in a powder state. In other embodiments, the salt of Formula (I) is in a crystalline state. In one embodiment, compound (1) is in a crystalline state. In other embodiments, the salt of Formula (I) is in a crystalline state which has various polymorphs. In one embodiment, compound (1) is in a crystalline state which has various polymorphs.

In some embodiments, Formula (I) as described herein is stable for 1-12 months. In one embodiment, Formula (I) salt is stable for 3-12 months. In another embodiment, Formula (I) salt is stable for 3-6 months. In another embodiment, Formula (I) salt is stable for 6-12 months. In another embodiment, Formula (I) salt is stable for 6-9 months. In another embodiment, Formula (I) salt is stable for 3-9 months. In another embodiment, Formula (I) salt is stable for 6 months. In another embodiment, compound (1) is stable for 3-12 months. In another embodiment, compound (1) is stable for 3-6 months. In another embodiment, compound (1) is stable for 6-12 months. In another embodiment, compound (1) is stable for 6-12 months. In another embodiment, compound (1) is stable for 6-9 months. In another embodiment, compound (1) is stable for 3-9 months. In another embodiment, compound (1) is stable for 6 months. In some other embodiments, compound (1) is stable for each of the described periods of time when it is found in a powder state. Each possibility represents a separate embodiment of the invention.

In one embodiment, without being bound by any mechanism or theory, it is herein contemplated that the high stability of the salts of the present invention is due to low and/or minimal hygroscopicity and/or high hydrophobic nature of the ammonium cations (Q, e.g. CHA). In another embodiment, the salts of Formula (I) are not hygroscopic. In another embodiment, the Q of Formula (I) is hydrophobic. In another embodiment, the salts of Formula (I) are not hygroscopic, and the Q of Formula (I) is hydrophobic. Each possibility represents a separate embodiment of the invention.

### Synthetic Applications of Formula (I)

In one embodiment, the present invention provides a method of preparing compound (3a) or (3b):
Step A:

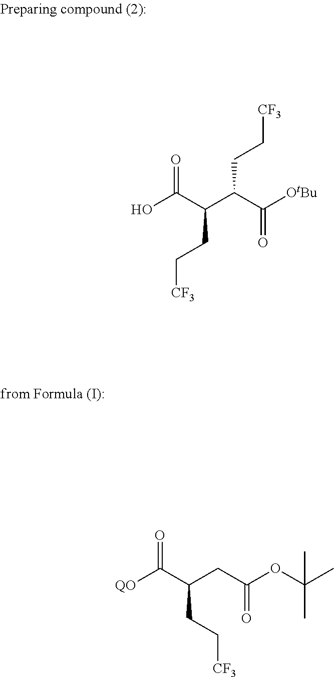

wherein
Q is an ammonium cation; and
Step B:

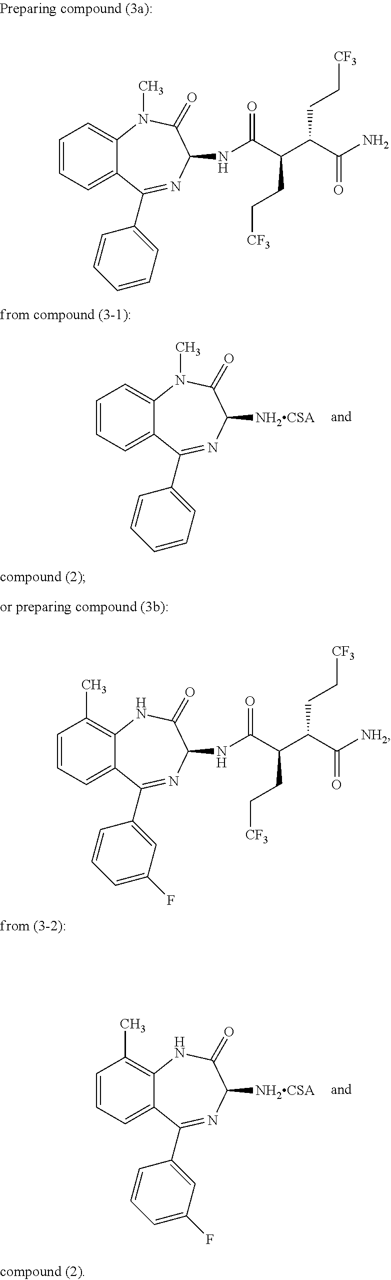

In another embodiment, the overall process is illustrated in scheme 1:

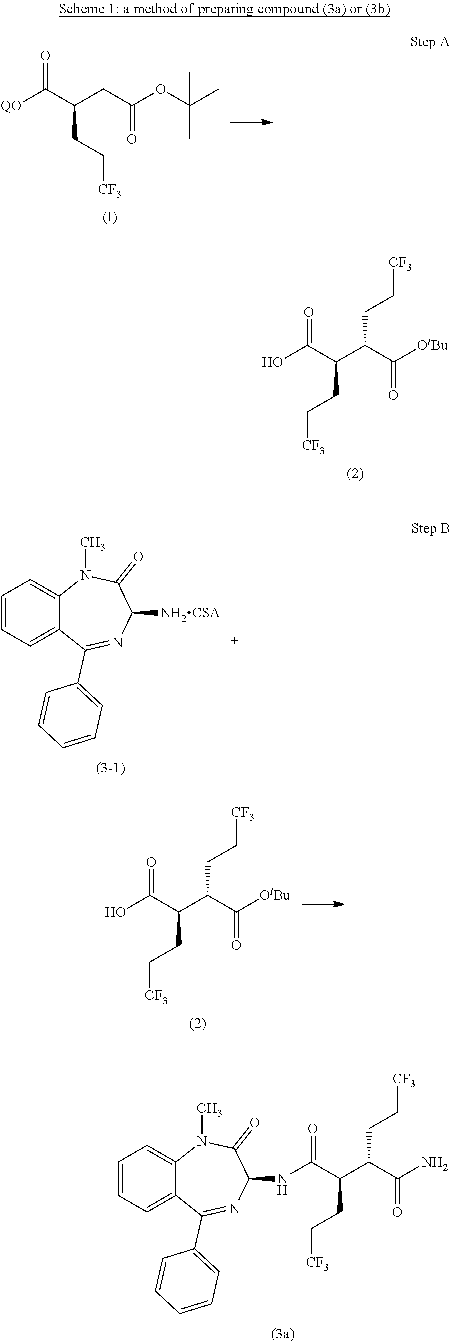

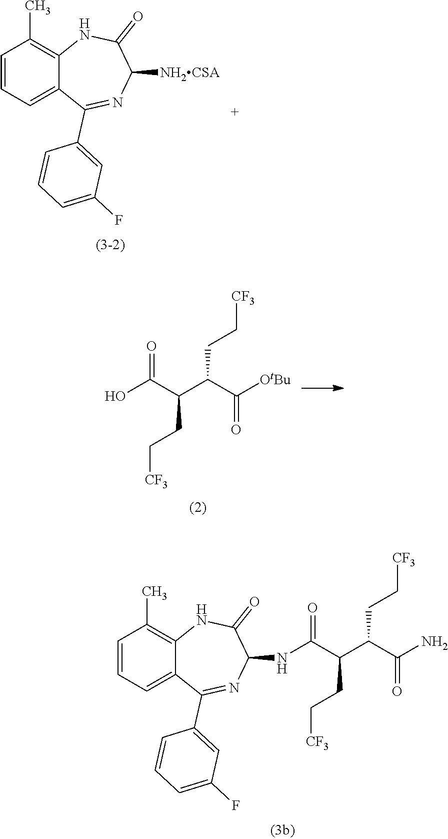

In another embodiment, Formula (I) is prepared via the following method, comprising:

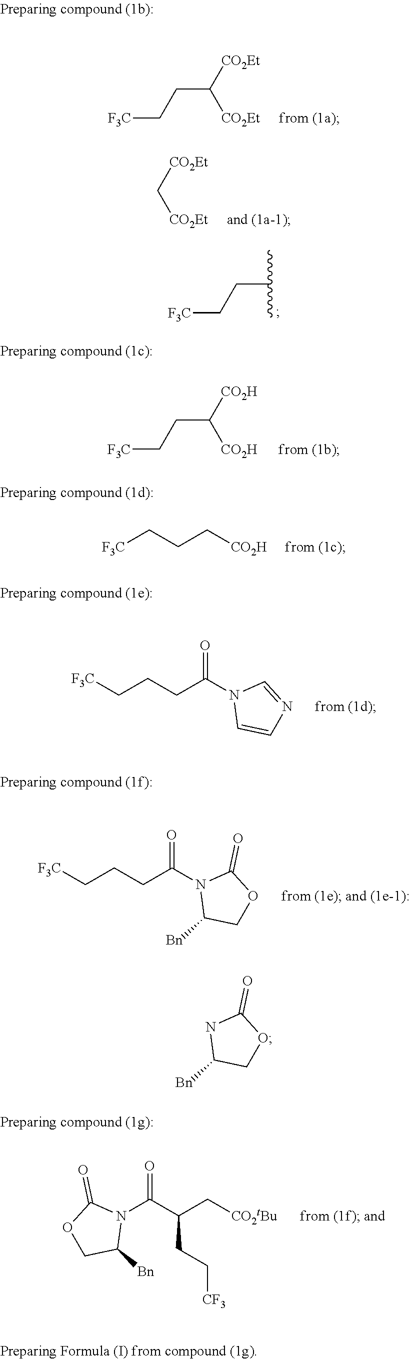

In another embodiment, the overall process is illustrated in scheme 2:

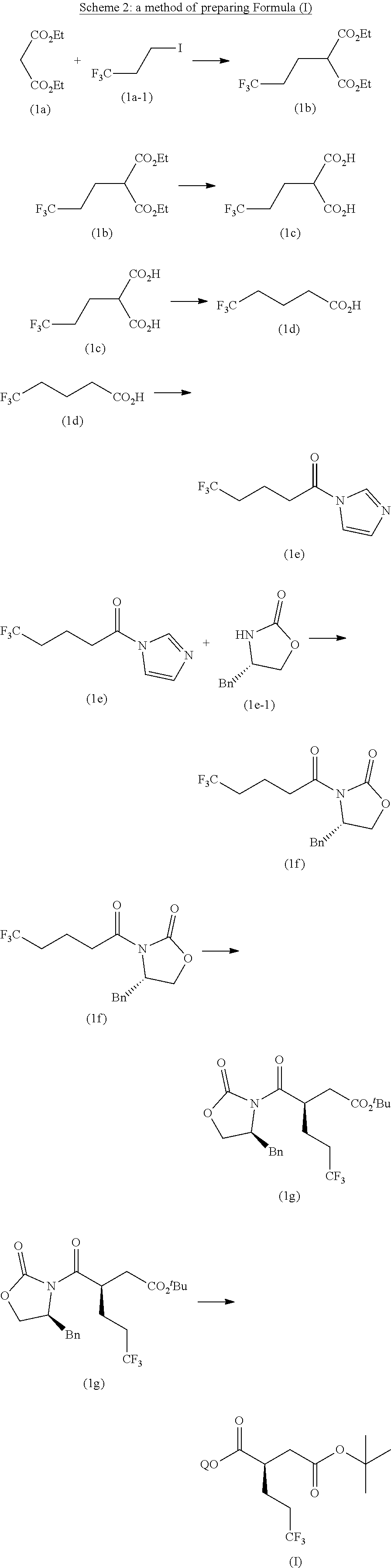

In another embodiment, (1b) is prepared from (1a) and (1a-1) under basic conditions. In another embodiment, NaOH is the base used. In another embodiment, (1a) is used in excess (when 1a-1 is the limiting reactant).

In another embodiment, (1c) is prepared from (1b) under basic conditions. In another embodiment, NaOH is the base used.

In another embodiment, (1d) is prepared from (1c) under acidic conditions. In another embodiment, the acid used is P-Toluene sulphonic acid.

In another embodiment, (1e) is prepared from (1d) under catalytic conditions. In another embodiment, the catalyst is DMAP (N,N-dimethyl-aminopyridine). In another embodiment, (1d) is reacted with imidazole coupling agent (e.g. 1,1′-carbonyldiimidazole) which is optionally in excess compared to (1d). In another embodiment, (1e) is prepared from (1d) under catalytic conditions (e.g. DMAP as catalyst) and (1d) is reacted with imidazole coupling agent (e.g. 1,1′-carbonyldiimidazole) which is optionally in excess compared to (1d).

In another embodiment, (1f) is prepared from (1e) under basic conditions. In another embodiment, the base is n-BuLi. In another embodiment, the reaction is performed at −78° C.

In another embodiment, (1g) is prepared from (1f) under basic conditions. In another embodiment, the base is NaHMDS (sodium-hexamethyldisilazane). In another embodiment, the reaction is performed at −78° C.

In another embodiment, the step of preparing Formula (I) from compound (1g) comprises:

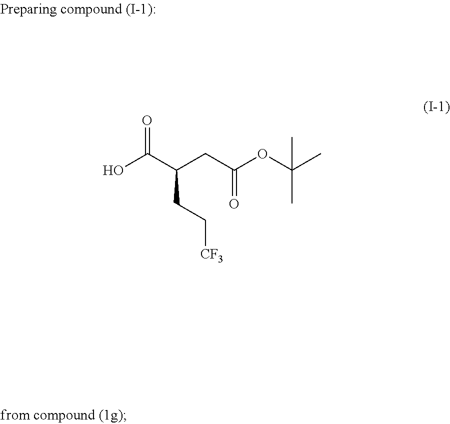 [SMILES: CC(C)(C)OC(=O)C[C@@H](CCC(F)(F)F)C(=O)O]

and
preparing Formula (I) from compound (I-1).

In another embodiment, the overall process is illustrated in scheme 2a:

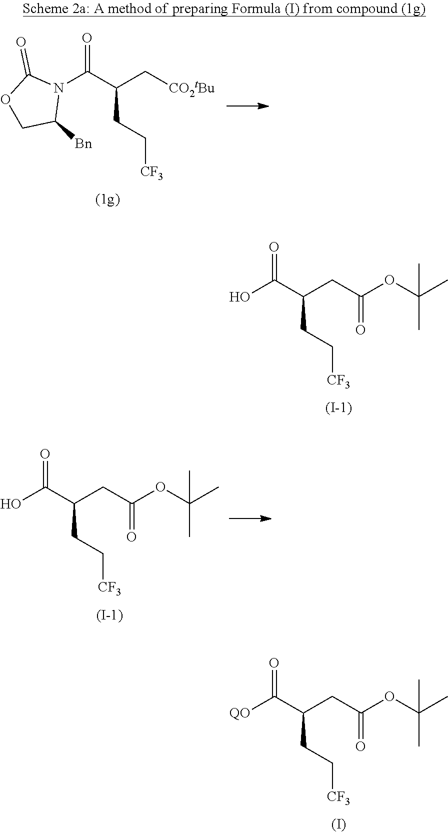

In a further embodiment, (1g) is reacted with an alkali metal peroxide (e.g. LiOOH, NaOOH, KOOH or CsOOH) or any other deprotecting group known in the art to remove the benzyloxazolidine-2-one moiety, to form (I-1).

In a further embodiment, (I-1) is reacted with an amine (e.g. CHA, to provide (1); or other amines—see examples as detailed above in Q), to form (I).

In another embodiment, compound (2) is prepared from Formula (I) via the following method, comprising:
Preparing compound (2a) (isomers mixture):

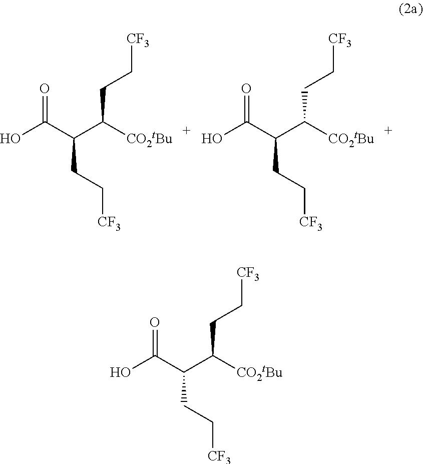 [SMILES: CC(C)(C)OC(=O)[C@H](CCC(F)(F)F)[C@@H](CCC(F)(F)F)C(=O)O ; CC(C)(C)OC(=O)[C@@H](CCC(F)(F)F)[C@@H](CCC(F)(F)F)C(=O)O ; CC(C)(C)OC(=O)[C@H](CCC(F)(F)F)[C@H](CCC(F)(F)F)C(=O)O]

(2a: R,R isomer) (2a: R,S isomer) (2a: S,R isomer)
from Formula (I); and compound (1g-1):

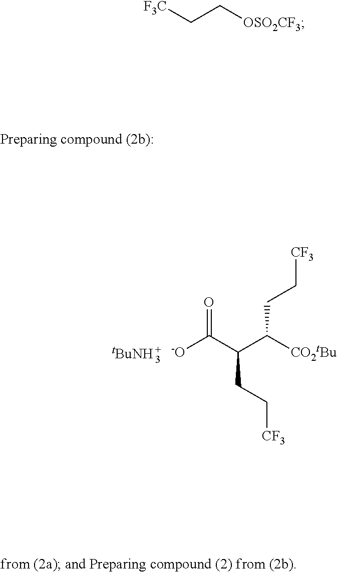

In one embodiment, the present invention provides a method of preparing compound (2) from Formula (I), comprising:
Preparing compound (2a) (isomers mixture):

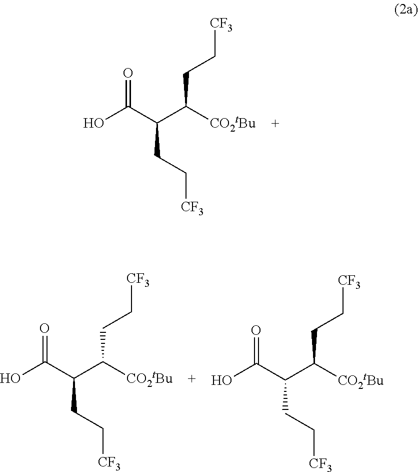 [SMILES: CC(C)(C)OC(=O)[C@H](CCC(F)(F)F)[C@@H](CCC(F)(F)F)C(=O)O ; CC(C)(C)OC(=O)[C@@H](CCC(F)(F)F)[C@@H](CCC(F)(F)F)C(=O)O ; CC(C)(C)OC(=O)[C@H](CCC(F)(F)F)[C@H](CCC(F)(F)F)C(=O)O]

(2a: R,R isomer) (2a: R,S isomer) (2a: S,R isomer)

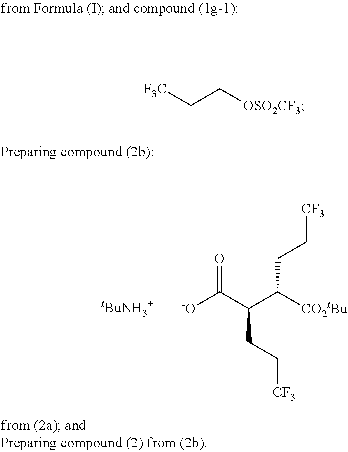

In some other embodiments, compound (1g-1) is prepared from compound (1g-11):

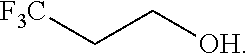

In another embodiment, (2a) further comprises an additional (S,S) isomer:

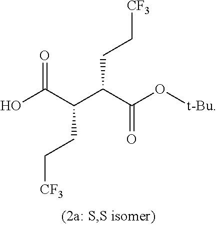 [SMILES: CC(C)(C)OC(=O)[C@@H](CCC(F)(F)F)[C@H](CCC(F)(F)F)C(=O)O]

In another embodiment, the overall process is illustrated in scheme 3:

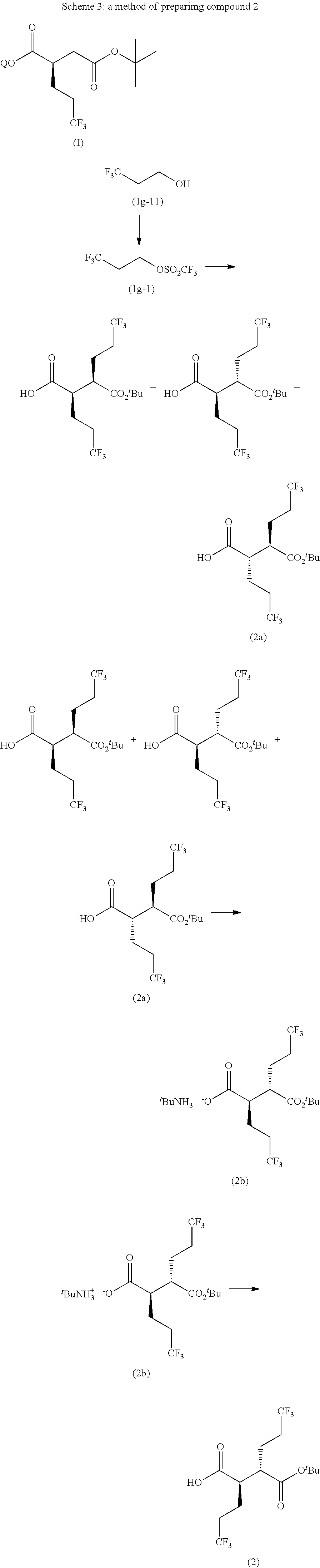

In another embodiment, (I) is first acidified to provide (I-1), before reacting with (1g-1). In another embodiment, the reaction of (I-1) with (1g-1) is performed under basic conditions. In another embodiment, the base is LDA (lithium-di-isopropylamide). In another embodiment, the reaction is performed at −78° C. and then heating to RT.

In another embodiment, the (R,R) and (R,S) isomers mixture of (2a) is not isolated. In another embodiment, tBuNH₂ reacts with (2a) to purify the (R,S) isomer from the (R,R) isomer, thus (R,R) isomer stays in the reaction mixture and is discarded; and the (2b) (i.e. (R,S) isomer of tBuNH₂ salt) is isolated (and finally provides (2) upon acidification). In another embodiment, the tBuNH₂ is used in the reaction in conjunction with LDA and CF₃CONH₂.

In another embodiment, compound (3-1) is prepared as detailed in Reider, P. J. et al., J. Org. Chem., 52:955-957 (1987), which is incorporated herein by reference.

In another embodiment, compound (3-2) is prepared as detailed in PCT publication number WO 2021/163676, which is incorporated herein by reference.

In another embodiment, compound (3a) is prepared from compounds (3-1) and (2) as detailed in PCT publication number WO 2012/129353, which is incorporated herein by reference.

In another embodiment, compound (3b) is prepared via the following method, comprising:

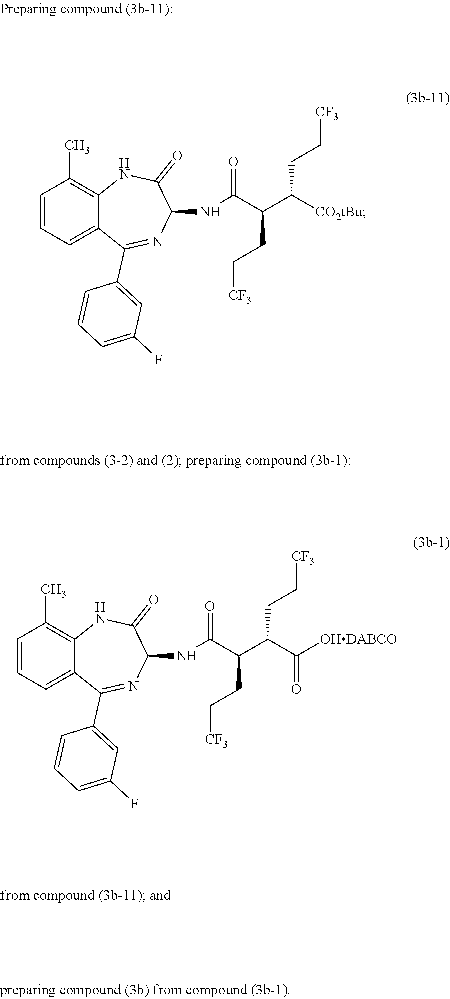

In another embodiment, the overall process is illustrated in scheme 4:

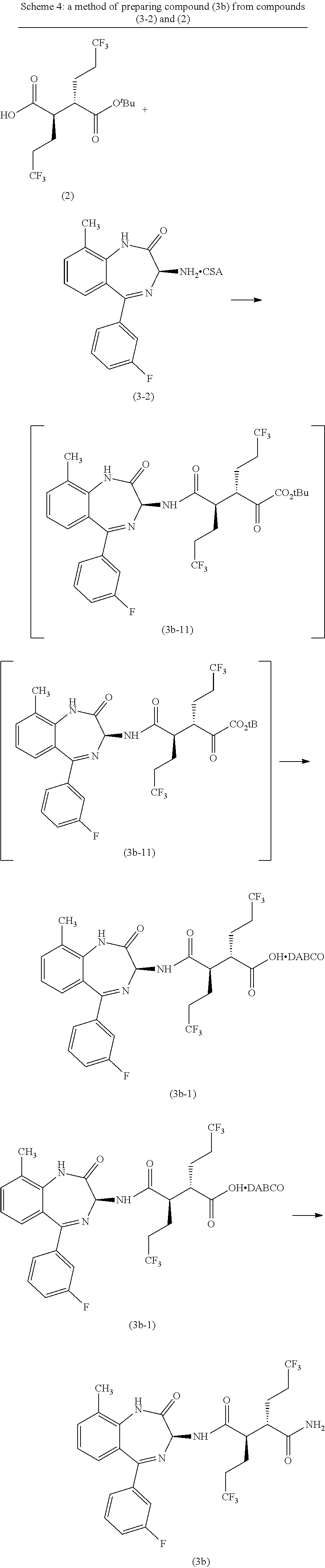

In another embodiment, (3b-11) is isolated. In another embodiment, (3b-11) is not isolated.

In another embodiment, (3b-11) is prepared from (2) and (3-2) under basic conditions. In another embodiment, the base is triethylamine. In another embodiment, the reaction is performed in the presence of a coupling reagent. In another embodiment, the coupling reagent is TBTU (2-(1H-Benzotriazole-1-yl)-1,1,3,3-tetramethylaminium tetrafluoroborate). In another embodiment, the reaction is performed in the presence of a base (e.g. triethylamine) and in the presence of a coupling reagent (e.g. TBTU).

In another embodiment, (3b-1) is prepared from (3b-11) upon acidification (with e.g. trifluoroacetic acid (TFA)) and subsequent basification with DABCO (1,4-diazabicyclo[2.2.2]octane).

In another embodiment, (3b) is prepared from (3b-1) upon amination (e.g. with ammonia) and a coupling agent (e.g. EDC (1-Ethyl-3-(3-dimethylaminopropyl)carbodiimide)). In another embodiment, the reaction further employs a coupling additive/enhancer such as HOBt (Hydroxybenzotriazole).

In some embodiments, any one of the intermediates presented within the methods of preparations of the present invention can be isolated or not (used without further purification).

In some embodiments, a solvent is used within the methods of the present invention. In one embodiment, the solvent comprises an alcoholic solvent, an ester solvent, an ether solvent, a hydrocarbon solvent, a polar aprotic solvent, a ketone solvent, a chlorinated solvent, a nitrile solvent, a polar solvent or any combination thereof. In another embodiment, non-limiting examples of alcoholic solvents include: methanol, ethanol, n-propanol, isopropanol, n-butanol, isobutanol, or a combination thereof. In another embodiment, non-limiting examples of ester solvents include: ethyl acetate, methyl acetate, isopropyl acetate, or a combination thereof. In another embodiment, non-limiting examples of ether solvents include: tetrahydrofuran, diethyl ether, methyl tert-butyl ether (MTBE), or a combination thereof. In another embodiment, non-limiting examples of hydrocarbon solvents include: toluene, hexane, heptane, cyclohexane, or a combination thereof. In another embodiment, non-limiting examples of polar aprotic solvents include: dimethyl acetamide, dimethyl formamide, dimethyl sulfoxide, or a combination thereof. In another embodiment, non-limiting examples of ketone solvents include: acetone, methyl ethyl ketone, methyl isobutyl ketone, or a combination thereof. In another embodiment, non-limiting examples of chlorinated solvents include: methylene chloride, chloroform, ethylene dichloride, or a combination thereof. In another embodiment, non-limiting examples of nitrile solvents include: acetonitrile, propionitrile, or a combination thereof. In another embodiment, non-limiting examples of polar solvents include water. In another embodiment, the solvent is ethanol. In another embodiment, the solvent is isopropyl acetate. Each possibility represents a separate embodiment of the invention.

In another embodiment, the reactions within the methods of the present invention are conducted at a temperature of between −5-100° C. In another embodiment, the temperature range is between 60-65° C. In another embodiment, the temperature range is between 0-45° C. In another embodiment, the temperature range is between −5-45° C. In another embodiment, the temperature range is between 5-10° C. In another embodiment, the temperature range is between −5-35° C. In another embodiment, the temperature range is between 0-35° C. In another embodiment, the temperature is 60° C. In another embodiment, the temperature range is between 0-100° C. Each possibility represents a separate embodiment of the invention.

### Definitions

In one embodiment, the term “stable for X weeks/months” (e.g. as in “the salts of Formula (I) are stable for 6-12 months”) means that said product (salts) keeps its initial purity and/or assay ±5% at least for the indicated time. In one embodiment, “keeping the initial purity of X at least for the indicated time” means that the purity should be kept approximately constant even beyond this indicated time (e.g., for a few additional months or years, which, in one embodiment, depends on the specific situation and storage conditions). In another embodiment, said stability is found in conditions of room temperature, 2-8° C. or 40° C. and 50-80, 50, 60, 70, 75 or 80% RH (relative humidity); or specifically at 2-8° C., 25° C./60% RH, or 40° C./75% RH. Each possibility and/or condition represents a separate embodiment of the invention.

In some embodiments, the term “room temperature” means a temperature of 10-40° C. In another embodiment, the term “room temperature” means a temperature of 15-35° C. In another embodiment, the term “room temperature” means a temperature of 25-35° C. In another embodiment, the term “room temperature” means a temperature of 20-30° C. In another embodiment, the term “room temperature” means a temperature of 10-20° C. In another embodiment, the term “room temperature” means a temperature of 10-30° C. Each possibility represents a separate embodiment of this invention.

In one embodiment, the term “reacting” within the context of the present invention is defined as provision of one or more conditions (e.g. heating, refluxing, mixing) which are sufficient for reactants to react chemically.

The following examples are presented in order to more fully illustrate the preferred embodiments of the invention. They should in no way, however, be construed as limiting the broad scope of the invention.

### EXAMPLES

### Example 1

### Synthesis of Compound (I-1)

LiOH·H₂O+H₂O₂→LiOOH·H₂O+H₂O

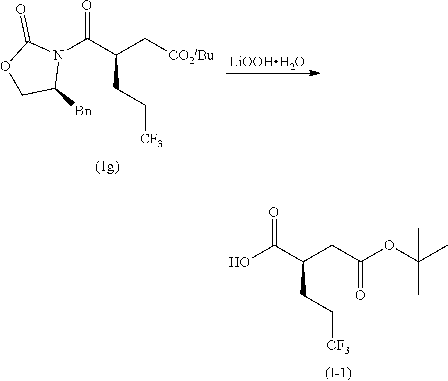

### Procedure

1. Water (7.5 L, 2 Vol) was charged into 600 L (HLR-111) reactor at 20-25° C.
2. LiOH·H₂O (0.557 Kg) was charged into 600 L reactor at 20-25° C., U/N₂.
3. The reaction mass was stirred for 5-10 min at 20-25° C.
4. The reaction mass was cooled to −5° C. to 0° C.
5. Hydrogen peroxide (30% w/v aqueous) (1.7 L, 1.7 eq) was added¹ into the reaction mass slowly below 5° C.
6. The reaction mass was stirred for 30 min at −5° C. to 0° C.
7. (R)-tert-butyl 3-((S)-4-benzyl-2-oxooxazolidine-3-carbonyl)-6,6,6-trifluorohexanoate ((1g); 3.8 Kg, Limiting Reagent) which is dissolved in THF (20 L, 5.3 Vol)² was added slowly into the reaction mass below 5° C.
8. The reaction mass was stirred for 1-2 hr at −5° C. to 0° C.
9. The reaction was monitored by HPLC (Limit: Assay: <8% w/w of (1g)). (Sampling: 2 ml of sample withdrawn from the reaction mass and submitted for in process HPLC analysis).
10. If beyond the limit, stirring was continued for 1 hr.
11. The reaction mass was quenched with aqueous sodium bi-sulfite solution (24.5%) (15.2 L, 4 Vol) at below 10° C. The pH of the aqueous solution was around 6˜5.5.
12. The reaction mass temperature was raised to 20-25° C.
13. The reaction mass was stirred for 30 min at 20-25° C.
14. The stirring was stopped, and the layers were allowed to separate.
15. The layers were separated: the bottom aqueous layer in HDPE-1 and top organic layer in HDPE-2.
16. The aqueous layer from the HDPE-1 was charged into the reactor.
17. Fresh MTBE (40 L, 10 Vol) was charged into the reactor.
18. The mixture was stirred for 10 min at RT.
19. The layers were separated: the bottom aqueous layer in HDPE-1 and top organic layer in HDPE-2.
20. The combined organic layer from the HDPE-2 was charged into the reactor.
21. Saturated sodium chloride solution (19 L, 5 Vol) was charged into the reactor.
22. The mixture was stirred for 10 min at RT.
23. The layers were separated: the bottom aqueous layer in HDPE-3 and organic layer in HDPE-4.
24. The organic layer from HDPE-4 was charged into the clean reactor.
25. The reaction mass was concentrated under vacuum at 45-50° C. up to 2.0 vol stage.
26. Fresh n-Heptane (11 L, 3 Vol) was charged into the reactor and concentration was continued up to 2 vol stage.
27. The solvent swap was repeated one more time with n-Heptane (11 L, 3 Vol) up to 2 Vol stage.
28. MTBE (3 L, 0.8 Vol) was charged into the reactor.
29. n-Heptane (19 L, 5 Vol) was charged into the reactor.
30. The reaction mass was cooled to 0-5° C. (solid was formed).
31. The slurry was stirred for 1 hr at below 0° C.
32. The solids were filtered using a clean Nutsche filter, and the wet cake was washed with n-Heptane (11 L, 3 Vol).
33. The material³ was unloaded into double polythene bags.
34. The filtrate was unloaded into clean HDPE-5 and labelled for its content.
35. The filtrate was charged from HDPE-5 into the clean reactor.
36. The reaction mass was concentrated under vacuum at 45-50° C. up to 2.0 Vol stage.
37. MTBE (38 L, 10 Vol) was charged into the reactor.
38. Petroleum ether (38 L, 10 Vol) was charged into the reactor.
39. The reaction mass was cooled to 15-20° C.
40. t-butyl amine (0.78 Kg, 1.2 eq) was added slowly into the reaction mass below 5° C. (solid was formed).
41. The slurry was stirred for 1-2 hr at 20-25° C.
42. The solids were filtered using a clean Nutsche filter, and the wet cake was washed with MTBE/Petroleum ether (1:1) mixture (12 L, 3 Vol).
43. The filtrate was transferred into clean HDPE-6 and labelled for its content.
44. The material was sucked dry for 1 hr at 20-25° C.
45. The material was unloaded into double polythene bags.
46. DCM (40 L, 10 Vol) was charged into 600 L (HLR-111) reactor at RT.
47. The above material (from step 44) was charged into the reactor.
48. The reaction mass was stirred for 5-10 min at 20-25° C.
49. 1.5 N HCl solution (30 L, 8 Vol) was charged into the reactor.
50. The reaction mass was stirred for 10 min at 20-25° C.
51. The bottom organic layer was separated in HDPE-7.
52. Fresh DCM (15 L, 4 Vol) was charged into the reactor.
53. The mixture was stirred for 10 min at RT.
54. The layers were separated: the bottom organic layer in HDPE-7 and top aqueous layer in HDPE-8.
55. The combined organic layer from the HDPE-7 was charged into the reactor.
56. Water (25 L, 6.5 Vol) was charged into the reactor.
57. The mixture was stirred for 10 min at RT.
58. The layers were separated: the bottom organic layer in HDPE-7 and top aqueous layer in HDPE-9.
59. The organic layer from the HDPE-7 was charged into the reactor.
60. Saturated sodium chloride solution (25 L, 6.5 Vol) was charged into the reactor.
61. The mixture was stirred for 10 min at RT.
62. The layers were separated: the bottom organic layer in HDPE-10 and top aqueous layer in HDPE-7.
63. Anhydrous sodium sulfate (1.0 w/w) was added into HDPE-10, and the organic layer was treated for about 1 hr.
64. The content of HDPE-10 was filtered using a clean Nutsche filter.
65. The filtrate was collected into HDPE-11.
66. The filtrate from the HDPE-11 was charged into the clean reactor.
67. The filtrate was concentrated under vacuum at 45-50° C. up to ˜1.0-1.5 Vol stage.
68. The material from the reactor was unloaded into a clean HDPE container and labelled for its content.
69. A small sample of the product was withdrawn and submitted for complete analysis.

### Notes

1. Addition of hydrogen peroxide (30%) was exothermic. Typically, 40 min taken for addition.
2. Addition of (1 g) solution was exothermic. Typically, 1.5 hr taken for addition.
3. Solid material was (S)-4-benzyloxazolidin-2-one

### Batch Summary

| Batch Input: | 3.8 | Kg |
|---|---|---|
| Batch Output: | 1.3 | Kg |
| Yield: | 54.36% | |
| AR No: | 95099-067-06 | |
| Appearance: | Yellowish liquid | |
| Purity by GC: | 98.38 | AP |
| Assay: | 97.60 | WP |

### Example 2

### Synthesis of CHA Salt (1)

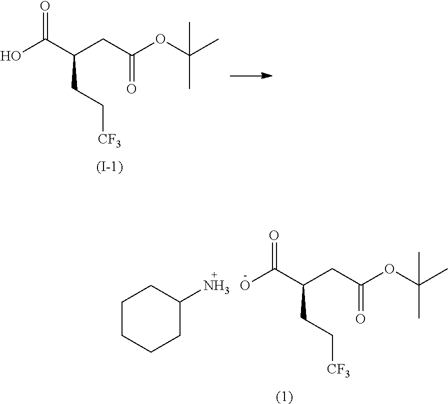

### Procedure

1. MTBE (10 w/w) was charged into (I-1) and stirred for 10 minutes at 25±5° C.
2. Cyclohexyl amine (0.252 w/w) was slowly added into the above reaction mass at 25±5° C. and stirred for not less than (NLT)) 30 min at 25±5° C.
3. The reaction mass temperature was raised to 45±5° C. and stirred for NLT 60 min at 45±5° C.
4. The reaction mass was cooled to 25±5° C. and stirred for NLT 60 min at 25±5° C.
5. The solid was filtered at 25±5° C., and the bed was washed with MTBE-com (5 w/w).
6. The material was sucked dry for NLT 30 Minutes, and the wet material was unloaded.
7. Wet material sample was sent to QC to check (1) HPLC purity and Chiral purity (By LCMS chiral purity method).
Acceptance criteria: HPLC Purity: NLT 90.0 A %
Chiral Purity: NLT 90.0 w/w %
HPLC Purity w/w: NLT 90.0 w/w %
Note: If results complied with the acceptance criteria—the material was subsequently purified.
8. MTBE-com (8 w/w) was charged followed by the wet material at 25±5° C.
9. The mixture was stirred for NLT 60 min at 25±5° C.
10. The solid was filtered, and the bed was washed with MTBE-com (5 w/w).
11. The material was sucked dry for NLT 30 minutes, and the wet material was unloaded.
12. Wet material sample was sent to QC to check (1) HPLC purity and Chiral purity (By LCMS chiral purity method).
Acceptance criteria: HPLC Purity: NLT 90.0 A %
Chiral Purity: NLT 90.0 w/w %
HPLC Purity: NLT 90.0 w/w %
Note: If results complied with the acceptance criteria—the material was subsequently purified.
13. The wet material was loaded into VTD (vacuum tray dryer) and dried for 2 hours at 25±5° C. under Vacuum (NLT 300 mm Hg).
Note: After 2 hours of drying, de-lumping and shuffling were performed.
14. The dryer temperature was raised to 45±5° C., and the material was dried in VTD under vacuum (NLT −300 mmHg) at 45±5° C. until the Water Content sample complied with acceptance criteria.
15. A sample was sent to QC for water content from 8^{th} hour onwards at every 4-hour interval until Water Content sample complied with the acceptance criteria.
   Note: Before taking a sample, the dry material is mixed.
   Acceptance criteria:
   Water Content: NMT 2.0% w/w
16. The VTD was cooled to 25±5° C., and the dried product was unloaded into double polyethylene bags placed in HDPE container.

### Example 3

### (I-1) and (1) Stability Study

Assay and water content of the oily free acid (I-1) were tested following a 6-month period in 25° C./60% RH (relative humidity) and 40° C./75% RH (Table 2a).

TABLE 2a
HPLC assay and water content of (I-1) initially and following a 6-
month period in 25° C./60% RH and 40° C./75% RH

| | Initial | | |
|---|---|---|---|
| Test | Results | 25° C./60% RH | 40° C./75% RH |
| Assay, % w/w | 93.5 | 92.8 | 84.5 |
| (by HPLC) | | | |
| Water content, % | 0.8 | 2.0 | 2.3 |
| (by KF) | | | |

Similarly, the corresponding CHA salt (1), which is a powder, was tested for its purity/assay:

TABLE 2b
HPLC assay and water content of (1) initially and following a 6-
month period in 25° C./60% RH and 40° C./75% RH:

| | Initial | | |
|---|---|---|---|
| Test | Results | 25° C./60% RH | 40° C./75% RH |
| Assay, % w/w | 95.0 | 95.1 | 94.4 |
| (by HPLC) | | | |
| Water content, % | 0.2 | 0.3 | 0.3 |
| (by KF) | | | |

TABLE 2c
HPLC assay of (1) initially and following a 1-, 3-, and 6-month period
in 25° C./60% RH, 40° C./75% RH and 2-8° C.:

Assay by HPLC, % w/w

| Condition | 2-8° C. | 25° C./60% RH | 40° C./75% RH |
|---|---|---|---|
| Initial | 95.0 | | |
| 1 month | 96.2 | 94.8 | 94.3 |
| 3 months | 95.1 | 94.5 | 93.0 |
| 6 months | 94.6 | 95.1 | 94.4 |

As can be seen, the salt (1) is much more stable compared to the free acid compound (I-1). The high purity assay of the salt does not practically change over time, in all tested conditions, whereas the free acid begins in an HPLC purity of ˜93% which is reduced further over time. It could be stipulated that increased water amount over time in the free acid gives rise to the observed assay which deteriorates as the test progresses.

### Example 4

### Stability of Na and tBuNH₂ Salts of (I-1)

Na and tBuNH₂ salts of (I-1) were prepared. Stability of the salts was tested and compared to the stability of the CHA salt ((1)).

TABLE 3
GC/MS assay of salts of (I-1) in various conditions

| | |
|---|---|
| Compound | Purity, % w/w |

| Salt of | After | At 2-8° C. | At 45° C. |
|---|---|---|---|
| (I-1) | synthesis | for 1 week | for 1 week |
| Na | 99.1 | 93.5 | 82.2 |
| CHA | — | 95.8 | 98.2 |
| (compound (1)) | | | |
| tBuNH₂ | 97.4 | 93.5 | 93.7 |

As illustrated in Table 3, CHA salt (compound (1)) was the most stable salt. This corresponds well with the results shown in Example 3 where the high purity of (1) did not practically change over time. It is also noted that the CHA salt (compound (1)) showed an increase in purity after heating, which could potentially be correlated to residual solvent evaporation during the heating. Table 3 also emphasizes that specific salts of (I-1) provide high purity over time. However, it cannot be said that any possible salt (any possible cation) of (I-1) will give rise to such high stability over time.

### Example 5

### Synthesis of Compound (2)

### a. (1b), (1c), and (1d) Preparation

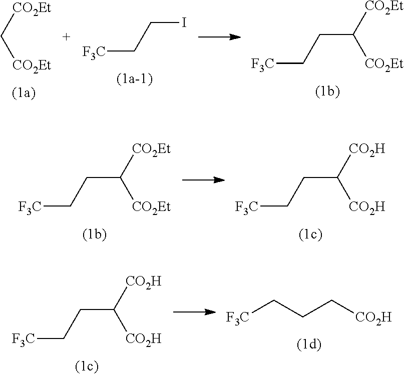

1. DMF (4.72 kg, 4.72 w/w) was charged into the reactor at 20 to 30° C.
2. Sodium hydroxide pellets (0.2 kg, 0.2 w/w) were charged into the reactor at 20 to 30° C.
3. The reaction mixture was stirred for NLT 10 min under nitrogen atmosphere.
4. The reaction mass temperature was raised to 40±5° C.
5. The reaction mixture was stirred for NLT 10 min under nitrogen atmosphere at 40±5° C.
6. (1a) (1.07 kg, 1.07 w/w) was charged to reaction mass at 40±5° C. over the period of NLT 30 min.
7. The reaction mass temperature was raised to 45±5° C.
8. The reaction mixture was stirred for NLT 60 min under nitrogen atmosphere at 45±5° C.
9. (1a-1) (1.00 kg, 1.00 w/w) was added slowly to reaction mass at 45±5° C. under nitrogen atmosphere.
10. The reaction mixture was stirred for NLT 60 min under nitrogen atmosphere at 45±5° C.
11. The sample was sent to QC from 1^{st} onwards every 2 h interval to check the (1a-1) content by GC until complies. Acceptance criteria: (1a-1) content-NMT (not more than) 3.0 A (area of integration) % by GC. Note: Sample preparation: About 5 mL of the reaction mass was drawn and submitted to QC.
12. Methanol:Water mixture (methanol (0.79 kg, 0.79 w/w) was added slowly and mixed in purified water (1.0 kg, 1.0 w/w)) through charging vessel into the reactor at 45±5° C.
13. 10N NaOH solution (NaOH (0.8 kg, 0.8 w/w) was slowly added and dissolved in purified water (2.0 kg, 2.0 w/w)) through charging vessel into the reactor at 45±5° C.
14. The mass temperature was raised to 50±5° C. under nitrogen atmosphere.
15. The reaction mixture was stirred for NLT 60 min under nitrogen atmosphere.
16. The sample was sent to QC from 1st h onwards every 2 h interval to check the (1b) content by GC until complies. Acceptance criteria: (1b) content-NMT 2.0 A % by GC.
Note: Sample preparation: About 5 mL of the reaction mass taken in a vial and quench the reaction mass with 2.5 mL of water and extracted with 5 mL of methyl tert-butyl ether (MTBE), submit the MTBE layer for GC analysis.
17. The reaction mass was cooled to 25±5° C.
18. Purified water (3.0 kg, 3.0 w/w) was added slowly into the reactor at 25±5° C.
19. Methyl tert-butyl ether (3.70 kg, 3.70 w/w) was charged into the reactor at 25±5° C.
20. The reaction mass was stirred for NLT 30 min at 25±5° C.
21. The reaction mass was allowed to settle for NLT 15 minutes.
22. The bottom aqueous layer was collected in a separate HDPE container.
23. The top organic layer was collected in a separate HDPE container.
24. The aqueous layer at step no. 22 was charged back into the reactor at 25±5° C.
25. Methyl tert-butyl ether (3.70 kg, 3.70 w/w) was charged into the above reactor at 25±5° C.
26. The reaction mass was stirred for NLT 30 min at 25±5° C.
27. The reaction mass was settled for NLT 15 minutes.
28. The bottom aqueous layer was collected in a separate HDPE container.
29. The top organic layer was collected in a separate HDPE container.
30. The aqueous layer at step no. 28 was charged back into the reactor through vacuum.
31. The reaction mass was cooled to 15±5° C.
32. 6N HCl solution (Conc. HCl (3.22 to 4.50 kg, 3.22 to 4.50 w/w)) was slowly added and mixed in purified water (2.28 to 3.20 kg to 2.28 to 3.20 w/w)) to reaction mass at 15±5° C.
Note: The pH of the reaction mass was adjusted to acidic (1.0-1.5).
33. Methyl tert-butyl ether (7.40 kg, 7.40 w/w) was charged into the reactor at 25±5° C.
34. The reaction mass was stirred for NLT 30 min at 25±5° C.
35. The reaction mass was allowed to settle for NLT 15 minutes.
36. The bottom aqueous layer was collected in a separate HDPE container.
37. The top organic layer was collected in a separate HDPE container.
38. The aqueous layer at step no. 36 was charged back into the reactor.
39. Methyl tert-butyl ether (3.70 kg, 3.70 w/w) was charged into the reactor at 25±5° C.
40. The reaction mass was stirred for NLT 30 min at 25±5° C.
41. The reaction mass was settled for NLT 15 minutes.
42. The bottom aqueous layer was collected in a separate HDPE container.
43. The top organic layer was collected in a separate HDPE container.
44. The aqueous layer at step no. 42 was charged back into the reactor through vacuum.
45. MTBE (3.70 kg, 3.70 w/w) was charged into the reactor at 25±5° C.
46. The reaction mass was stirred for NLT 30 min at 25±5° C.
47. The reaction mass was allowed to settle for NLT 15 minutes.
48. The bottom aqueous layer was collected in a separate HDPE container, and the organic layer was kept in the reactor.
49. The organic layers of step nos 37 & 43 were charged back into reactor at 25±5° C.
50. Water (5.0 kg, 5.0 w/w) was charged into the reactor at 25±5° C.
51. The reaction mass was stirred for NLT 30 min at 25±5° C.
52. The reaction mass was allowed for NLT 15 minutes.
53. The bottom aqueous layer was collected in a separate HDPE container, and the organic layer was kept in the reactor.
54. Water (5.0 kg, 5.0 w/w) was charged into the reactor at 25±5° C.
55. The reaction mass was stirred for NLT 30 min at 25±5° C.
56. The reaction mass was allowed to settle for NLT 15 minutes.
57. The bottom aqueous layer was collected in a separate HDPE container, and the organic layer was kept in the reactor.
58. Water (5.0 kg, 5.0 w/w) was charged into the reactor at 25±5° C.
59. The reaction mass was stirred for NLT 30 min at 25±5° C.
60. The reaction mass was allowed to settle for NLT 15 minutes.
61. The bottom aqueous layer was collected in a separate HDPE container, and the organic layer was kept in the reactor.
62. Water (5.0 kg, 5.0 w/w) was charged into the reactor at 25±5° C.
63. The reaction mass was stirred for NLT 30 min at 25±5° C.
64. The reaction mass was allowed to settle for NLT 15 minutes.
65. The bottom aqueous layer was collected in a separate HDPE container, and the organic layer was kept in the reactor.
66. Sodium sulfate (0.5-1.0 kg, 0.5-1.0 w/w) was charged into the reactor.
67. The reaction mass was stirred for NLT 30 min at 25±5° C.
68. The mass was filtered by using a Nutsche filter under vacuum.
69. The bed was washed with Methyl tert-butyl ether (2.25 kg, 2.25 w/w) at 25±5° C.
70. The filtrate was collected in HDPE container.
71. The filtrate of step no. 70 was charged into the clean and dry reactor at 25±5° C.
72. The mass was concentrated at below 50° C. under vacuum until no distillate is observed, and the high vacuum was applied for NLT 60 minutes.
73. The sample was sent to QC to check the (1c) content by HPLC. Acceptance criteria: (1c) content result was reported.
74. Dimethyl formamide (4.20 kg, 4.20 w/w) was charged into the reactor at below 50° C., and the mass was concentrated at below 50° C. under vacuum NLT 45 min.
75. The reaction mass was cooled to 25±5° C.
76. P-Toluene sulphonic acid monohydrate (1.20 kg, 1.20 w/w) was charged into the reactor at 25±5° C.
77. The reaction mixture was stirred for NLT 10 min under nitrogen atmosphere at 25±5° C.
78. The mass temperature was raised to 105±5° C.
79. The reaction mass was stirred at 105±5° C. for 4 h, the sample was then sent to QC from 4^{th} hours onwards every 2 hours interval to check the (1c) content by HPLC assay until the reaction complies. Acceptance criteria: (1c) content-NMT 1.0 w/w % by HPLC.
Note: Sample preparation: About 5 mL of the reaction mass was drawn and submitted to QC.
80. The reaction mass temperature was cooled to 20±5° C.
81. Purified water (8.90 kg, 8.90 w/w) was slowly added into the reactor 20±5° C.
82. The reaction mass temperature was raised to 25±5° C.
83. Methyl tert-butyl ether (6.6 kg, 6.6 w/w) was charged to the reactor at 25±5° C.
84. The reaction mass was stirred for NLT 30 min at 25±5° C.
85. The reaction mass was allowed to settle for NLT 15 minutes.
86. The bottom aqueous layer was collected in a separate HDPE container.
87. The top organic layer was collected in a separate HDPE container.
88. The aqueous layer at step no. 86 was charged back into the reactor at 25±5° C.
89. Methyl tert-butyl ether (6.6 kg, 6.6 w/w) was charged to the reactor 25±5° C.
90. The reaction mass was stirred for NLT 30 min at 25±5° C.
91. The reaction mass was allowed to settle for NLT 15 minutes.
92. The bottom aqueous layer was collected in a separate HDPE container the organic layer in was kept in the reactor.
93. The organic layer was charged back into the reactor from step no. 87.
94. Water (8.90 kg, 8.90 w/w) was charged into the reactor at 25±5° C.
95. The reaction mass was stirred for NLT 30 min at 25±5° C.
96. The reaction mass was allowed to settle for NLT 15 minutes.
97. The bottom aqueous layer was collected in separate HDPE container, and the organic layer was kept in the reactor.
98. Water (8.90 kg, 8.90 w/w) was charged into the reactor at 25±5° C.
99. The reaction mass was stirred for NLT 30 min at 25±5° C.
100. The reaction mass was allowed to settle for NLT 15 minutes.
101. The bottom aqueous layer was collected in separate HDPE container, and the organic layer was kept in the reactor.
102. Purified water (8.90 kg, 8.90 w/w) was charged into the reactor at 25±5° C.
103. The reaction mass was stirred for NLT 30 min at 25±5° C.
104. The reaction mass was allowed to settle for NLT 15 minutes.
105. The bottom aqueous layer was collected in separate HDPE container, and the organic layer was kept in the reactor.
106. Water (8.90 kg, 8.90 w/w) was charged into the reactor at 25±5° C.
107. The reaction mass was stirred for NLT 30 min at 25±5° C.
108. The reaction mass was allowed to settle for NLT 15 minutes.
109. The bottom aqueous layer was collected in separate HDPE container, and the organic layer was kept in the reactor.
110. Sodium sulfate (0.5 to 1.0 kg, 0.5-1.0 w/w) was charged into the reactor.
111. The reaction mass was stirred for NLT 30 min at 25±5° C.
112. The mass was filtered by using a Nutsche filter under vacuum.
113. The bed was washed with Methyl tert-butyl ether (2.25 kg, 2.25 w/w) at 25±5° C.
114. The filtrate was collected in HDPE container.
115. The filtrate at step no. 114 was charged into the reactor at 25±5° C.
116. The mass was concentrated at below 50° C. under vacuum until no distillate was observed, and high vacuum was applied for NLT 180 minutes.
117. The reaction mass was cooled to 30±5° C. The mass was unloaded into the HDPE container.
118. A sample was sent to QC for purity of (1d) by GC. Acceptance criteria: (1d) content-NLT 80.0% area by GC.

| | | |
|---|---|---|
| Yield: | 0.49 | kg |
| Theoretical yield: | 0.696 | kg |

| | |
|---|---|
| % Yield: | 70.40% |

### b. (1e) and (1f) Preparation

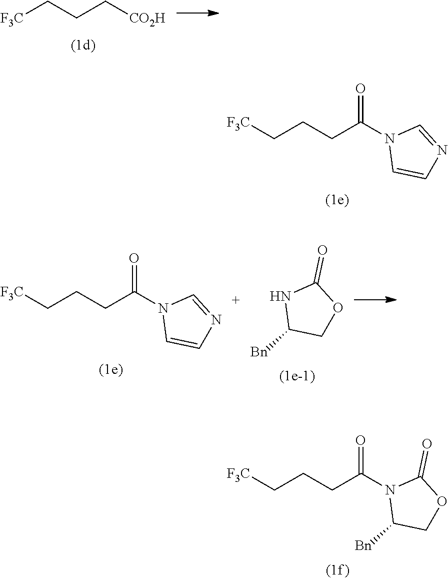 [SMILES: O=C(O)CCCC(F)(F)F ; O=C(CCCC(F)(F)F)n1ccnc1 ; O=C(CCCC(F)(F)F)n1ccnc1 ; O=C1N[C@@H](Cc2ccccc2)CO1 ; O=C(CCCC(F)(F)F)N1C(=O)OC[C@@H]1Cc1ccccc1]

1. THF-Dry (4.44 kg, 4.44 w/w) was charged into the reactor at 25±5° C.
10 ml of Tetrahydrofuran sample were sent to QC to check the water content by KF (Karl Fischer titration). Acceptance criteria: Water content NMT 0.2 w/v %.
2. (1d) (1.23 kg, 1.23 w/w) was charged into the reactor under nitrogen atmosphere at 25±5° C.
3. The reaction mixture was stirred for NLT 5 min under nitrogen atmosphere.
4. 4-(dimethylamino) pyridine (0.103 kg, 0.103 w/w) was charged into the reaction mass at 25±5° C.
5. The reaction mixture was stirred for NLT 5 min under nitrogen atmosphere.
6. N, N′-Carbonyldiimidazole was charged in portions (1.47 kg, 1.47 w/w) into the reaction mass at 25±5° C.
7. The reaction mixture was stirred for NLT 60 min under nitrogen atmosphere at 25±5° C.
8. The sample was sent to QC from 1st hour onwards every 2 h interval to check the (1d) content by GC until it complies. Acceptance criteria: (1d) content—NMT 2.0% area by GC.
9. The reaction mass was unloaded into a clean HDPE drum under nitrogen atmosphere.
10. THF-Dry (8.88 kg, 8.88 w/w) was charged into another reactor at 25±5° C. under nitrogen atmosphere.
10 ml of Tetrahydrofuran sample were sent to QC to check the water content by KF. Acceptance criteria: Water content NMT 0.2 w/v %.
11. (1e-1) (1.0 kg, 1.0 w/w) was added into the reaction under nitrogen atmosphere at 25±5° C.
12. The reaction mixture was stirred for NLT 5 minutes under nitrogen atmosphere at 25±5° C.
13. The reaction mass was cooled at −74±5° C. under nitrogen atmosphere.
14. n-Butyl lithium (2.5 M in hexane) (1.72 kg, 1.72 w/w) was added into the reaction mass slowly from cylinder at (−74)±5° C. under nitrogen atmosphere.
15. The reaction mass was stirred for NLT 120 min at −74±5° C. under nitrogen atmosphere.
16. The above-prepared solution (step no. 9) was added slowly through charging vessel to the reaction mass by maintaining the temperature at −74±5° C. under nitrogen atmosphere.
17. The reaction mass was stirred for 120 minutes at −74±5° C. under nitrogen atmosphere.
18. The sample was sent to QC from 2nd h onwards every 2 h interval to check the (1e-1) content by HPLC until it complies. Acceptance criteria: (1e-1)—NMT 5.0% area by HPLC.
Note: Sample preparation: About 5 mL of the reaction mass was taken in a vial, and the reaction mass was quenched with 5 mL aqueous ammonium chloride solution (20%), and the top organic layer was sent for HPLC.
19. The reaction mass temperature was raised to (−40)±10° C. under nitrogen atmosphere.
20. 30% ammonium chloride solution (Ammonium chloride (1.5 kg, 1.5 w/w) was added and dissolved in purified water (5.0 kg, 5.0 w/w) at −40±10° C. under nitrogen atmosphere.
21. The mass reaction temperature was raised to 25±5° C.
22. Methyl tert-butyl ether (7.40 kg, 7.40 w/w) was charged into the reaction mass at 25±5° C.
23. The reaction mass was stirred for NLT 15 min at 25±5° C.
24. The reaction mass was allowed to settle for NLT 15 minutes.
25. The bottom aqueous layer was collected in a separate HDPE container.
26. The top organic layer was collected in a separate HDPE container.
27. The aqueous layer (step no. 25) was charged back into the reactor at 25±5° C.
28. Methyl tert-butyl ether (7.40 kg, 7.40 w/w) was charged into the reaction mass at 25±5° C.
29. The reaction mass was stirred for NLT 15 min at 25±5° C.
30. The reaction mass was allowed to settle for NLT 15 minutes.
31. The bottom aqueous layer was collected in a separate HDPE container, and the organic layer was kept in the reactor.
32. The organic layer (step no. 26) was charged back into the reactor.
33. 10% Sodium bicarbonate solution (Sodium bicarbonate (1.0 kg, 1.0 w/w) was charged and dissolved in purified water (10.0 kg, 10.0 w/w) into above organic layer at 25±5° C.
34. The reaction mass was stirred for NLT 15 min at 25±5° C.
35. The reaction mass was allowed to settle for NLT 15 minutes.
36. The bottom aqueous layer was collected in a separate HDPE container, and the organic layer was kept in the reactor.
37. Purified water (10.0 kg, 10.0 w/w) was charged into the reactor at 25±5° C.
38. The reaction mass was stirred for NLT 15 min at 25±5° C.
39. The reaction mass was allowed to settle for NLT 15 minutes.
40. The bottom aqueous layer was collected in a separate HDPE container, and the organic layer was kept in the reactor.
41. 1.5 N HCl solution (Conc. HCl (1.61 kg, 1.61 w/w)) was slowly added and dissolved in purified water (8.64 w/w)) through charging vessel to the reactor at 25±5° C.
42. The reaction mass was stirred for NLT 15 min at 25±5° C.
43. The reaction mass was allowed to settle for NLT 15 minutes.
44. The bottom aqueous layer was collected in a separate HDPE container, and the organic layer was kept in the reactor.
45. Purified water (10.0 kg, 10.0 w/w) was charged into the reactor at 25±5° C.
46. The reaction mass was stirred for NLT 15 min at 25±5° C.
47. The reaction mass was allowed to settle for NLT 15 minutes.
48. The bottom aqueous layer was collected in a separate HDPE container, and the organic layer was kept in the reactor.
49. Sodium chloride solution (Sodium chloride (2.8 kg, 2.8 w/w)) was charged and dissolved in purified water (10.0 kg, 10.0 w/w) into above organic layer at 25±5° C.
50. The reaction mass was stirred for NLT 15 min at 25±5° C.
51. The reaction mass was allowed to settle for NLT 15 minutes.
52. The bottom aqueous layer was collected in a separate HDPE container, and the organic layer was kept in the reactor.
53. Sodium sulfate (1.0 kg, 1.0 w/w) was charged into the above organic layer at 25±5° C.
54. The reaction mass was stirred for NLT 30 min at 25±5° C.
55. The mass was filtered using a Nutsche filter under vacuum.
56. The bed was washed with Methyl tert-butyl ether (2.22 kg, 2.22 w/w) at 25±5° C.
57. The filtrate was collected in HDPE container.
58. The filtrate (step no. 57) was charged into the clean and dry reactor at 25±5° C.
59. The mass was concentrated at below 50° C. under vacuum until no distillate is observed.
60. The n-Heptane (2.05 kg, 2.05 w/w) was charged into the reactor at below 50° C.
61. The mass was concentrated at below 50° C. under vacuum until no distillate is observed.
62. n-Heptane (2.05 kg, 2.05 w/w) was charged into the reactor at below 50° C.
63. The mass was concentrated at below 50° C. under vacuum until no distillate is observed, and the high vacuum was applied for NLT 60 minutes.
64. The reaction mass was cooled to 25±5° C.
65. The sample was sent to QC for the purity of (1f) by HPLC.
66. The reactor was rinsed with Ethyl acetate (0.902 kg, 0.902 w/w) at 25±5° C.
67. n-Heptane (6.16 kg, 6.16 w/w) was added slowly into the reaction mass at 25±5° C.
68. The reaction mass was cooled at below 20° C.
69. (1f) was charged as seeding material (0.010 kg, 0.010 w/w) into the reaction mass at below 20° C.
70. The reaction mass was cooled to 0±5° C.
71. The reaction mixture was stirred for NLT 1 h at 0±5° C.
72. The reactor was rinsed with n-Heptane (3.42 kg, 3.42 w/w) at 0±5° C.
73. The bed was washed with the above n-Heptane solvent at 0±5° C.
74. The material was sucked dry for NLT 60 minutes, and the wet material was unloaded into double polythene bags and placed in HDPE container.
75. The wet sample was sent to QC for the purity of (1f) by HPLC and water content. Acceptance criteria: (1f) content—NLT 90.0% area by HPLC and water content.

| | | |
|---|---|---|
| Yield: | ~1.16 | kg |
| Theoretical yield: | 2.02 | kg |

| | |
|---|---|
| % Yield: | ~57.42% |

### c. (1g), (I-1), and (1) Preparation

### (1g)→(1) is Performed in an Alternative Procedure to the One of Examples 1-2

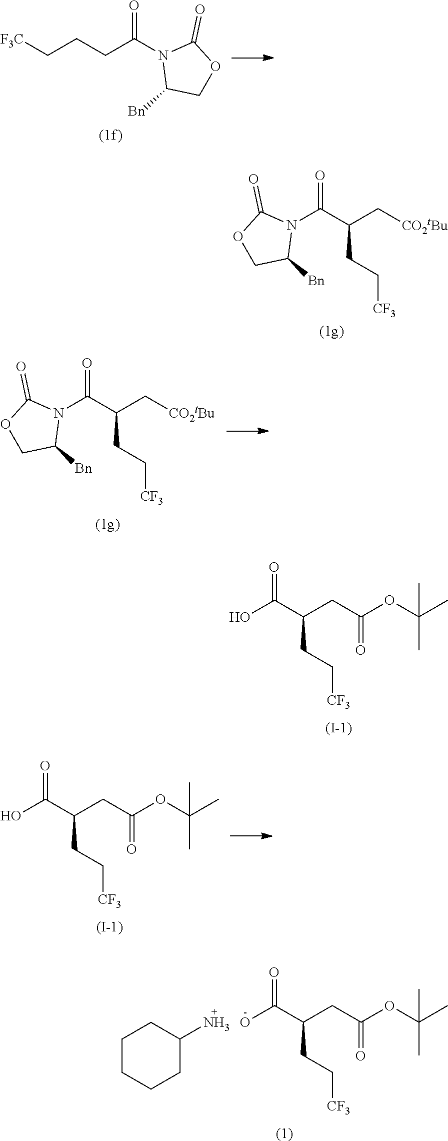

1. THF-Dry (8.89 kg, 8.89 w/w) was charged into the reactor at 25±5° C. under nitrogen atmosphere.
10 ml of Tetrahydrofuran sample was sent to QC to check the water content by KF. Acceptance criteria: Water content NMT (not more than) 0.15 w/v %.
2. (1f) (1.0 kg, 1.0 w/w) was charged into the reactor, and the reaction mixture was stirred for NLT 5 min under nitrogen atmosphere.
5 ml reaction mass sample was sent to QC to check the water content by KF. Acceptance criteria: Water content NMT 0.15 w/v %.
3. Molecular sieves 4a powdered (0.5 kg, 0.5 w/w) were charged into the reactor at 25±5° C.
4. The reaction mixture was stirred for NLT 30 min under nitrogen atmosphere at 25±5° C.
The sample was sent to QC from 30 minutes onwards every 1 h interval to check water content by KF until complies. Acceptance criteria: Water content NMT 0.15 w/v %.
5. Preparation of celite bed: Celite (0.7-1.5 kg, 0.7-1.5 w/w) and THF-dry (0.89-1.78 kg, 0.89-1.78 w/w) were charged in HDPE container. The celite solution was filtered through sparkler filter. The solvent was unloaded and sent for disposal.
6. The reaction mass (step no. 4) was filtered through the above sparkler filter under nitrogen atmosphere.
7. The wet cake was washed with THF-dry (0.89-1.78 kg, 0.89-1.78 w/w), and the filtrate was transferred to another reactor through nitrogen pressure with closed condition.
5 ml sample was sent from the reactor (at step no. 7) to QC to check the water content by KF. Acceptance criteria: Water content NMT 0.15 w/v %.
8. The reaction mass was cooled to −70±5° C. under nitrogen atmosphere.
9. NaHMDS (1M solution in THF) (3.15 w/w) was added to charging vessel under vacuum under nitrogen atmosphere.
10. NaHMDS (1M solution in THF) (3.15 kg, 3.15 w/w) was slowly added into the reaction mass temperature −70±5° C. over the period of NLT 45 minutes under nitrogen atmosphere.
11. The reaction mixture was stirred for NLT 2 h at −70±5° C. under nitrogen atmosphere.
12. Tert-butyl bromoacetate (0.805 kg, 0.805 w/w) was slowly added into the reaction mass at −70±5° C. over the period of NLT 30 min under nitrogen atmosphere.
13. The reaction mass was stirred for NLT 60 min under nitrogen atmosphere.
14. The sample was sent to QC from 1st hour onwards every 2 hour interval to check (1f) content by HPLC until complies. Acceptance criteria: (1f) content—NMT 5.0 A % by HPLC.
Note: Sampling: 2 ml of reaction mass was quenched with 2 ml of 20% NH4CI solution and extracted with 2 ml of EtOAc; the ethyl acetate layer was submitted for analysis.
15. The reaction mass temperature was raised to 0±10° C. under nitrogen atmosphere.
16. 20% ammonium chloride solution (Ammonium chloride (2.0 kg, 2.0 w/w)) was slowly added and dissolved in purified water (10.0 kg, 10.0 w/w)) into the reaction mass at 0±10° C. under nitrogen atmosphere.
17. The reaction mass temperature was raised to 25±5° C.
18. The Ethyl acetate (9.02 kg. 9.02 w/w) was charged into the reactor at 25±5° C.
19. The reaction mass was stirred for NLT 15 min at 25±5° C.
20. The reaction mass was allowed to settle for NLT 15 minutes.
21. The bottom aqueous layer was collected in a separate HDPE container, and the organic layer was kept in the reactor.
22. Charge 10% sodium bi carbonate solution (sodium bi carbonate (1.0 kg, 1.0 w/w) dissolve in purified water (10.0 kg, 10.0 w/w)) into above organic layer at 25±5° C.
23. The reaction mass was stirred for NLT 15 min at 25±5° C.
24. The reaction mass was allowed to settle for NLT 15 minutes.
25. The bottom aqueous layer was collected in a separate HDPE container, and the organic layer was kept in the reactor.
26. Sodium chloride solution (sodium chloride (2.8 kg, 2.8 w/w) was charged and dissolved in purified water (10.0 kg, 10.0 w/w)) into above organic layer at 25±5° C.
27. The reaction mass was stirred for NLT 15 min at 25±5° C.
28. The reaction mass was allowed to settle for NLT 15 minutes.
29. The bottom aqueous layer was collected in a separate HDPE container.
30. The organic layer was collected in a separate HDPE container.
31. The organic layer at (step no. 30) was charged back into the clean and dry reactor at 25±5° C.
32. The organic layer was concentrated at below 50° C. under vacuum until no distillate is observed, and high vacuum was applied for NLT 60 minutes.
33. The reaction mass was cooled to 25±5° C.
34. The sample was sent to QC for purity of (1g) by HPLC.
35. THF (5.0 kg, 5.0 w/w) was charged into the reactor under vacuum, and the mass was stirred for NLT 10 min at 25±5° C.
36. The reaction mass was unloaded into HDPE container.
37. The reactor was rinsed with THF (1.50 kg, 1.50 w/w) at 25±5° C.
38. The above rinsed THF was unloaded into the above HDPE container (at step no. 36).
39. 5 ml sample were sent to QC for assay of (1g) by HPLC.
40. Purified water (2.73 kg, 2.73 w/w) was charged into the clean and dry reactor at 25±5° C.
41. Lithium hydroxide monohydrate (0.20 kg, 0.20 w/w) was charged into the reactor at 25±5° C.
42. The reaction mass was stirred for NLT 10 min at 25±5° C.
43. The reaction mass was cooled to 0±5° C.
44. Hydrogen peroxide (30%) (0.61 kg, 0.61 w/w) was slowly added into reaction mass at 0±5° C.
45. The reaction mass was stirred for NLT 30 min at 0±5° C.
46. The above prepared (1g) in THF solution (step no. 38) was added slowly into reaction mass through at 0±5° C.
47. The reaction mass was stirred for NLT 60 min at 0±5° C.
48. The sample was sent to QC for content of (1g) by HPLC assay from 2^{nd} hour onwards at every 3 hour until it complied. Acceptance criteria: (1g) content: NMT 1.0 w/w %. Note: Sampling: 2 ml of reaction mass was quenched with 2 ml of 25% sodium bi sulphite, and the top THF layer was submitted for analysis.
49. 25% Sodiumbisulphite solution (Sodiumbisulphite (1.0 kg, 1.0 w/w dissolve in purified water (4.0 kg, 4.0 w/w)) was slowly added into the reaction mass at below 10° C.
50. The reaction mass temperature was raised to 25±5° C.
51. The reaction mass was stirred for NLT 15 min at 25±5° C.
52. The reaction mass was allowed to settle for NLT 15 minutes.
53. The bottom aqueous layer was collected in a separate HDPE container. Check the pH of the aqueous layer using pH meter (5-7).
54. The top organic layer was collected in a separate HDPE container.
55. The aqueous layer (step no. 53) was charged back into the reactor at 25±5° C.
56. Methyl tert-butyl ether (10.0 kg, 10.0 w/w) was charged to the reactor at 25±5° C.
57. The reaction mass was stirred for NLT 15 min at 25±5° C.
58. The reaction mass was allowed to settle for NLT 15 minutes.
59. The bottom aqueous layer was collected in a separate HDPE container, and the organic layer was kept in the reactor.
60. The organic layer (step no. 54) was charged back into the reactor at 25±5° C.
61. 25% Sodiumbisulphite solution (Sodiumbisulphite (1.0 kg, 1.0 w/w dissolve in purified water (4.0 kg, 4.0 w/w)) was added into the reaction mass at below 25±5° C.
62. The reaction mass was stirred for NLT 15 min at 25±5° C.
63. The reaction mass was allowed to settle for NLT 15 minutes.
64. The bottom aqueous layer was collected in a separate HDPE container, and the organic layer was kept in the reactor.
65. 5 ml organic layer was sent to QC to check peroxide content. Acceptance criteria: Peroxide content: NMT 25 ppm
66. Sodium chloride solution (Sodium chloride (1.4 kg, 1.4 w/w dissolve in purified water (5.0 kg, 5.0 w/w)) was added into the organic layer at 25±5° C.
67. The reaction mass was stirred for NLT 15 min at 25±5° C.
68. The reaction mass was allowed to settle for NLT 15 minutes.
69. The bottom aqueous layer was collected in a separate HDPE container, and the top organic layer was collected in a separate HDPE container.
70. The organic layer (step no. 69) was charged into a clean and dry reactor.
71. The mass was concentrated at below 50° C. under vacuum until no distillate is observed.
72. The n-Heptane (3.72 kg, 3.72 w/w) was charged into the reactor at below 50° C.
73. The mass was concentrated at below 50° C. under vacuum until no distillate is observed.
74. The n-Heptane (3.72 kg, 3.72 w/w) was charged into the reactor at below 50° C.
75. The mass was concentrated at below 50° C. under vacuum until no distillate is observed, and high vacuum was applied for NLT 60 minutes.
76. The reaction mass was cooled to 25±5° C.
77. Methyl tert-butyl ether (0.81 kg, 0.81 w/w) was charged into the reactor at 25±5° C.
78. n-Heptane (2.80-4.65 kg, 2.80-4.65 w/w) was charged into the reactor.
79. The reaction mass was cooled to 0±5° C.
80. The reaction mass was stirred for NLT 60 minutes at 0±5° C.
81. The solid was filtered through the Nutsche filter, and the filtrate was collected into the HDPE containers.
82. n-Heptane (2.79-3.50 kg, 2.79-3.50 w/w) was charged into the reactor.
83. The solvent was cooled to 0±5° C.
84. The bed was washed with above solvent (at step no. 83).
85. The filtrate was collected to the same HDPE Container (at step no. 81).
86. The material was sucked dry for NLT 30 minutes, and the wet material was unloaded into double polythene bags and was sent for disposal (By product is (S)-(−)-4-benzyl-2-oxazolidinone).
87. The filtrate (at step no. 85) was charged into a clean reactor at 25±5° C.
88. The filtrate was concentrated at below 50° C. under vacuum until no distillate is observed.
89. The reaction mass was cooled to 25±5° C.
90. The sample was sent to QC to check purity of (I-1) by GC.
91. Methyl tert-butyl ether (7.4-10.0 kg, 7.4-10.0 w/w) was charged into the reactor at 25±5° C.
92. Cyclohexyl amine (0.252 kg, 0.252 w/w) was slowly added into the reaction mass at 25±5° C.
93. The reaction mass was stirred for NLT 30 min at 25±5° C.
94. The reaction mass temperature was raised to 45±5° C.
95. The reaction mass was stirred for NLT 60 min at 45±5° C.
96. The reaction mass was cooled to 25±5° C.
97. The reaction mass was stirred for NLT 60 min at 25±5° C.
98. The solid was filtered through the Nutsche filter at 25±5° C. under vacuum.
99. The material was sucked dry for NLT 30 minutes, and the wet material was unloaded into double polythene bags and was placed in HDPE container.
100. The wet material sample was sent to QC to check (1) HPLC purity and Chiral purity (By LCMS). Acceptance criteria: HPLC Purity: NLT 91.0 w/w % and Chiral Purity (other isomer): NMT 1.0 w/w %
101. If results complied with the acceptance criteria, then step no. 110 was the next step; otherwise, step no. 102 (MTBE purification) was the next.
102. The Methyl tert-butyl ether (10.0 w/w, based on wet weight at step no. 99) was charged into the reactor.
103. The wet material (at step no. 99) was charged into the reactor at 25±5° C.
104. The wet material (at step 103) was stirred for NLT 60 min at 25±5° C.
105. The solid was filtered through the Nutsche filter at 25±5° C. under vacuum.
106. The bed was washed with Methyl tert-butyl ether (3.0 w/w, based on wet weight at step no. 99).
107. The material was sucked dry for NLT 30 minutes, and the wet material was unloaded into double polythene bags and was placed in HDPE container.
108. The wet material sample was sent to QC to check (1) HPLC purity and Chiral purity (By LCMS). Acceptance criteria: HPLC Purity: NLT 91.0 w/w % and Chiral Purity (other isomer): NMT 1.0 w/w %.
109. If results complied with the acceptance criteria, then step no. 110 was the next step; otherwise, step no. 102 (MTBE purification) was the next.
110. The wet material from step number 99/107 was loaded into VTD (vacuum tray dryer) and dried for 2 hours at 25±5° C. under vacuum (NLT 300 mm Hg).
111. The vacuum dryer temperature was raised to 45±5° C.
112. The material was dried in VTD under vacuum (NLT −300 mmHg) at 45±5° C. until the residual content and water content sample complied with acceptance criteria.
A sample was sent to QC for water content from 8^{th} hour onwards at every 4-hour interval until residual content and water content complied with the acceptance criteria.
Acceptance criteria: Water Content: NMT 0.8% w/w.
Residual solvents (By GC-HS)
   Methyl tert-butyl ether—NMT 4000 ppm
   Tetrahydrofuran—NMT 720 ppm
   Ethyl acetate—NMT 5000 ppm
   n-Heptane—NMT 5000 pppm
113. The VTD was cooled to 25±5° C.
114. The dried product was unloaded into double polyethylene bags placed in HDPE container.

| | | |
|---|---|---|
| Yield: | ~0.5 | kg |
| Theoretical yield: | 1.17 | kg |

| | |
|---|---|
| % Yield: | ~42.73% |

d. (1g-1) Preparation

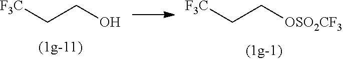

1. Dichloromethane (13.3 kg, 13.30 w/w) was charged into a reactor at 25±5° C. under nitrogen atmosphere.
Dichloromethane sample was sent to QC for water content by KF (Acceptance criteria NMT (not more than) 0.20% w/v).
2. 3,3,3-trifluoro-1-propanol ((1g-11); 1.0 kg, 1.0 w/w) was charged into a reactor at 25±5° C. under nitrogen atmosphere.
3. 2,6 Lutidine (1.42 kg, 1.42 w/w) was charged into a reaction mass at 25±5° C. under nitrogen atmosphere.
4. The reaction mass was stirred for NLT 5 min at 25±5° C. under nitrogen atmosphere.
5. The reaction mass was cooled to −25±5° C. under nitrogen atmosphere.
6. Trifluoromethane sulfonic anhydride (3.46 kg, 3.46 w/w) was slowly added into reaction mass at −25±5° C. under nitrogen atmosphere.
7. The reaction mass was stirred for NLT 120 min at −25±5° C. under nitrogen atmosphere.
8. The reaction mass temperature was slowly raised to 20±5° C. under nitrogen atmosphere.
9. The reaction mass was stirred for 1 hr at 20±5° C. under nitrogen atmosphere.
A sample was sent to QC for 3,3,3-trifluoro-1-propanol (1g-11) content by GC from 1^{st} hour onwards at every 2 hour interval until 3,3,3-trifluoro-1-propanol content was reduced to a sufficient level.
Acceptance criteria: 3,3,3-trifluoro-1-propanol content NMT 5.0%.
Note: Sampling procedure: About 5 mL of the reaction mass was drawn and submitted to QC.
10. The reaction mass was cooled to 0±5° C. under nitrogen atmosphere.
11. Purified water (7.0 kg, 7.0 w/w) was slowly added into reaction mass at 0±5° C. under nitrogen atmosphere.
12. The reaction mass was stirred for NLT 15 min at 0±5° C.
13. The reaction mass temperature was slowly raised to 20±5° C.
14. The reaction mass was stirred for NLT 15 min at 20±5° C.
15. The mass was allowed to settle for NLT 15 min.
16. The bottom organic layer was separated into the HDPE container, and the aqueous layer was collected in a separate container.
17. The organic layer was charged back into reactor, and purified water (7.0 kg, 7.0 w/w) was added at 20±5° C.
18. The reaction mass was stirred for NLT 15 min at 20±5° C.
19. The mass was allowed to settle for NLT 15 min.
20. The bottom organic layer was separated into HDPE container, and the aqueous layer was collected in separate container.
21. The organic layer was charged back into reactor and concentrated completely under vacuum at below 30° C. until ˜2.5-4.0 Vol of mass remained inside the reactor.
22. The above crude was fractionally distilled under high vacuum at below 40° C. and different fractions were collected.

(1g-1)

| | | |
|---|---|---|
| Yield: | ~1.2 | kg |
| Theoretical yield: | 2.15 | kg |

| | |
|---|---|
| % Yield: | ~55.81% |

### e. (I-1), (2a) and (2b) Preparation

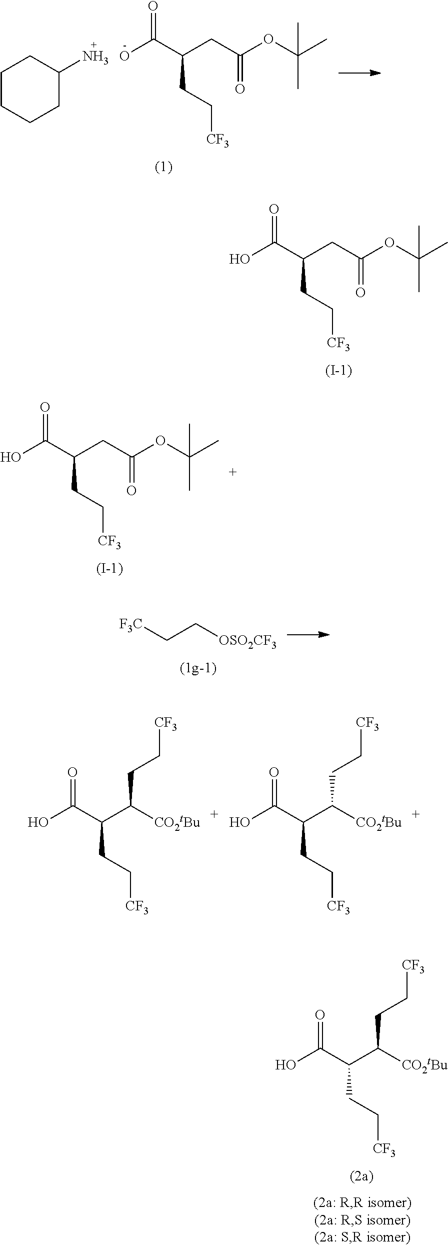

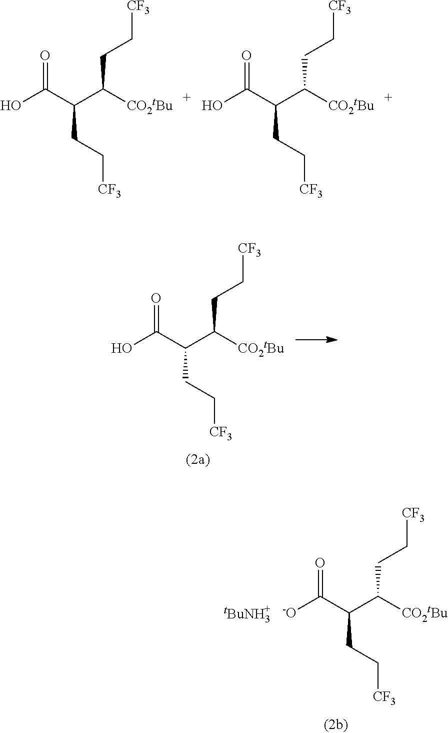

1) Dichloromethane (18.10 kg, 18.10 w/w) was charged into the reactor at below 30° C.
2) Compound (1) (1.0 kg, 1.0 w/w) was charged into the reactor at below 30° C.
3) The reaction mass was stirred for NLT 10 min at 25±5° C.
4) 1.5 N HCl solution (conc. HCl (1.58 kg, 1.58 w/w) was slowly added and mixed in purified water (8.42 kg, 8.42 w/w)), and the resulting solution was added to the reaction mass at 25±5° C.
5) The reaction mass was stirred for NLT 10 min at 25±5° C.
6) The mass was allowed to settle for NLT 10 min at 25±5° C.
7) The bottom organic layer was separated in clean HDPE container. The aqueous layer was kept in the reactor.
8) The Dichloromethane (7.24 kg, 7.24 w/w) was charged into the above aqueous layer at 25±5° C.
9) The reaction mass was stirred for NLT 10 min at 25±5° C.
10) The mass was allowed to settle for NLT 10 min 25±5° C.
11) The bottom organic layer was separated in clean HDPE container, and the top aqueous layer in a clean HDPE drum.
12) The combined organic layer (step nos. 7 & 11) was charged back into the reactor at 25±5° C.
13) The purified water (9.0 kg, 9.0 w/w) was charged into the above organic layer at 25±5° C.
14) The reaction mass was stirred for NLT 15 min at 25±5° C.
15) The mass was allowed to settle for NLT 15 min at 25±5° C.
16) The bottom organic layer was separated in a clean HDPE drum, and the top aqueous layer in a clean HDPE drum.
17) The above organic layer (step no. 16) was charged back into reactor at 25±5° C.
18) The saturated brine solution (NaCl (2.17 kg, 2.17 w/w) was charged and dissolved in purified water (7.83 w/w)) into above organic layer at 25±5° C.
19) The reaction mass was stirred for NLT 10 min at 25±5° C.
20) The mass was allowed to settle for NLT 10 min at 25±5° C.
21) The bottom organic layer was separated in a clean HDPE drum and top aqueous layer in a clean HDPE drum.
22) The above organic layer (step no. 21) was charged back into the reactor at 25±5° C.
23) Sodium sulfate (2.0 kg, 2.0 w/w) was charged into above organic layer at 25±5° C.
24) The reaction mass was stirred for NLT 15 min at 25±5° C.
25) The reaction mass was filtered through a Nutsche filter, and the filtrate was collected into the clean HDPE containers.
26) The reactor was rinsed with Dichloromethane (4.0 kg, 4.0 w/w), and the wet cake was washed, and the filtrate was collected in HDPE containers.
27) The combined above organic layer was charged into a clean and dry reactor at 25±5° C.
28) The reaction mass was concentrated completely under vacuum at below 45° C.
29) The reaction mass was cooled to 25±5° C. and unloaded in HDPE container. (Wt of (I-1): ˜0.7 kg).
All raw materials input based on weight at step no. 29 ((I-1)).
30) Tetrahydrofuran-Dry (6.23 kg, 8.90 w/w) was charged into the reactor under nitrogen atmosphere at 25±5° C.
THF sample was sent to QC for water content by KF (Acceptance criteria NMT 0.10% w/v).
31) Diisopropyl amine (0.682 to 0.76 kg, 0.974 to 1.08 w/w) was added into reaction mass under nitrogen atmosphere at 25±5° C.
32) The reaction mixture was stirred for NLT 5 min at below 30° C. under nitrogen atmosphere.
33) The reaction mass was cooled to −45±10° C. for NLT 30 minutes.
34) n-Butyl lithium 2.5M was slowly added in hexane (1.87 to 2.17 kg, 2.67 to 3.10 w/w) into the reaction mass from the cylinder at −45±10° C. under nitrogen atmosphere.
35) The reaction mass was stirred at −45±10° C. for NLT 5 min under nitrogen atmosphere.
36) The reaction mass temperature was slowly raised to 0±10° C. under nitrogen atmosphere.
37) The reaction mixture was stirred for NLT 40 min at 0±10° C. under nitrogen atmosphere.
38) The reaction mass was cooled to −75±8° C. for NLT 30 minutes under nitrogen atmosphere.
39) The (I-1) solution (0.7 kg, 1.0 w/w) was slowly added as solution of THF-Dry (3.11 kg, 4.45 w/w) into the reaction mass at −75±8° C. under nitrogen atmosphere.
40) The reaction mixture was stirred for NLT 120 min at −75±8° C. under nitrogen atmosphere.
41) (1g-1) (1.085 kg, 1.55 w/w) was slowly added to reaction mass at −75±8° C. under nitrogen atmosphere over a period of NLT 30 minutes.
42) The reaction mixture was stirred for NLT 2 hour at −75±8° C. under NITROGEN atmosphere.
43) The sample was sent to QC to check the (I-1) content by HPLC (w/w %) from 2^{nd} hour onwards at every 2-hour interval up to 8^{th} hour sample (Acceptance criteria: (I-1) content NMT 25.0 w/w %).
Note: Sample procedure: 5 mL of sample was taken from the reaction mass and quenched with 20% Ammonium chloride solution, and the organic layer was separated and submitted as an IPC (in process control).
44) The reaction mass temperature was slowly raised to −30±10° C. under nitrogen atmosphere.
45) The above prepared 20% ammonium chloride solution (Ammonium chloride (0.7 kg, 0.7 w/w dissolved in purified water (3.5 kg, 5.0 w/w)) was slowly added into the reaction mass through a charging vessel by at below 0° C.
46) The reaction mass temperature was raised to 25±5° C.
47) Methyl tert-butyl ether-com (10.36 to 11.90 kg, 14.8 to 17.0 w/w) was charged to the reactor 25±5° C.
48) The reaction mass was stirred for NLT 15 min at 25±5° C.
49) The mass was allowed to settle for NLT 15 min at 25±5° C.
50) The bottom aqueous layer was separated in a clean HDPE container, and the organic layer was kept in the reactor.
51) Purified water (3.5 kg, 5.0 w/w) was charged into the above organic layer at below 30° C.
52) The reaction mass was stirred for NLT 15 min at 25±5° C.
53) The mass was allowed to settle for NLT 15 min at 25±5° C.
54) The bottom aqueous layer was separated in a clean HDPE container, and the organic layer was kept in the reactor.
55) Purified water (3.5 kg, 5.0 w/w) was charged into the above organic layer at below 30° C.
56) The reaction mass was stirred for NLT 15 min at 25±5° C.
57) The mass was allowed to settle for NLT 15 min at 25±5° C.
58) The bottom aqueous layer was separated in a clean HDPE container, and the organic layer was kept in the reactor.
59) Purified water (3.5 kg, 5.0 w/w) was charged into the above organic layer at below 30° C.
60) The reaction mass was stirred for NLT 15 min at 25±5° C.
61) The mass was allowed to settle for NLT 15 min at 25±5° C.
62) The bottom aqueous layer was separated in a clean HDPE container, and the organic layer was kept in the reactor.
63) The organic layer was cooled to 20±5° C.
64) The above prepared 1.5 N HCl solution (Conc. HCl (1.12 to 1.68 kg, 1.60 to 2.4 w/w)) was slowly added and mixed in purified water (5.88 to 8.82 kg, 8.4 to 12.6 w/w) to organic layer through charging vessel at 20±10° C., and the reaction mass pH was adjusted to below 3.0
65) The reaction mass was stirred for NLT 15 min at 25±5° C.
66) The mass was allowed to settle for NLT 15 min at 25±5° C.
67) The bottom aqueous layer was separated in a clean HDPE container, and the organic layer was kept in the reactor.
68) Purified water (4.9 kg, 7.0 w/w) was charged into the above organic layer at below 30° C.
69) The reaction mass was stirred for NLT 15 min at 25±5° C.
70) The mass was allowed to settle for NLT 15 min at 25±5° C.
71) The bottom aqueous layer was separated in a clean HDPE container, and the organic layer was kept reactor.
72) Purified water (4.9 kg, 7.0 w/w) was charged into the above organic layer at below 30° C.
73) The reaction mass was stirred for NLT 15 min at 25±5° C.
74) The mass was allowed to settle for NLT 15 min at 25±5° C.
75) The bottom aqueous layer was separated in a clean HDPE container, and the organic layer was kept in the reactor.
76) Sodium sulfate (0.7 kg, 1.0 w/w) was charged into the above organic layer at 25±5° C.
77) The reaction mass was stirred for NLT 15 min at 25±5° C.
78) The reaction mass was filtered through a Nutsche filter, and the filtrate was collected into the HDPE container.
79) The reactor was rinsed with Methyl tert-butyl ether (1.4 to 2.8 kg, 2.0 to 4.0 w/w), and the wet cake was washed, and the filtrate was collected in HDPE container.
80) The above organic layer was charged back into a clean and dry reactor and was concentrated completely under vacuum at below 55° C.
81) The reaction mass was cooled to 25±5° C. and unloaded into HDPE container under nitrogen atmosphere (Weight of (2a): ˜0.77 kg).
The sample was sent to QC to check the (I-1) content by HPLC (w/w %) Acceptance criteria: Content of (I-1) (By HPLC): NMT 10.0 (w/w)
All RMs input based on weigh at step no. 81 (2a)
82) Tetrahydrofuran-Dry (6.85 kg, 8.90 w/w) was charged into the reactor under nitrogen atmosphere at 25±5° C.
THF sample was sent to QC for water content by KF (Acceptance criteria NMT 0.10% w/v).
83) Diisopropyl amine (0.53 to 0.60 kg, 0.69 to 0.78 w/w) was added into reaction mass under nitrogen atmosphere at 25±5° C.
84) The reaction mixture was stirred for NLT 5 min at below 30° C. under nitrogen atmosphere.
85) The reaction mass was cooled to −45±10° C. for NLT 30 minutes.
86) n-Butyl lithium 2.5M in hexane (1.40 to 1.71 kg, 1.82 to 2.22 w/w) was slowly added into the reaction mass from the cylinder at −45±10° C. under nitrogen atmosphere.
87) The reaction mass was stirred at −45±10° C. for NLT 5 min under nitrogen atmosphere.
88) The reaction mass temperature was slowly raised to 0±10° C. under nitrogen atmosphere.
89) The reaction mixture was stirred for NLT 30 min at 0±10° C. under nitrogen atmosphere.
90) The reaction mass was cooled to −75±8° C. for NLT 30 minutes under nitrogen atmosphere.
91) The (2a) solution (0.77 kg, 1.0 w/w) dissolved in THF-Dry (3.46 kg, 4.45 w/w) was slowly added into the reaction mass at −75±8° C. under nitrogen atmosphere.
92) The reaction mixture was stirred for NLT 3 hour at −75±8° C. under nitrogen atmosphere.
93) The reaction mass was cooled to −100±10° C. under nitrogen atmosphere.
94) 2,2,2-Trifluoroacetamide solution (0.57 kg, 0.74 w/w dissolved in THF-Dry (1.04 kg, 1.35 w/w)) was slowly added into the reaction mass through charging vessel at −100±10° C. under nitrogen atmosphere.
95) The reaction mass was stirred at −100±10° C. for NLT 120 min under nitrogen atmosphere.
96) The reaction mass temperature was slowly raised to −30±10° C. under nitrogen atmosphere.
97) 20% ammonium chloride solution (Ammonium chloride (0.46 kg, 0.6 w/w) dissolved in purified water (2.31 kg, 3.0 w/w)) was slowly added to reaction mass at through charging vessel at −30±10° C.
98) The reaction mass temperature was raised to 20±10° C.
99) 1.5 N HCl solution (Conc. HCl (1.12 to 1.68 kg, 1.60 to 2.4)) was mixed in purified water (5.88 to 8.82 kg, 8.40 to 12.6 w/w) and slowly added to reaction mass by charging vessel at below 20±10° C. The pH of the reaction mass was adjusted to acidic below 3.
100) Methyl tert-butyl ether (5.7 kg, 7.4 w/w) was charged into the above reaction mass at below 30° C.
101) The reaction mass was stirred for NLT 15 min at 25±5° C.
102) The mass was allowed to settle for NLT 15 min at 25±5° C.
103) The bottom aqueous layer was separated in clean HDPE container, and the top organic layer was separated in clean HDPE container.
104) The aqueous layer (step no. 103) was charged back into the reactor at 25±5° C.
105) Methyl tert-butyl ether (2.85 kg, 3.7 w/w) was charged into the above aqueous layer at below 30° C.
106) The reaction mass was stirred for NLT 15 min at 25±5° C.
107) The mass was allowed to settle for NLT 15 min at 25±5° C.
108) The bottom aqueous layer was separated in clean HDPE container, and the organic layer was kept in the reactor.
109) Purified water (7.7 kg, 10.0 w/w) was charged into the above organic layer at below 30° C.
110) The reaction mass was stirred for NLT 5 min at 25±5° C.
111) 0.5 N NaOH solution (NaOH (0.015 to 0.15 kg, 0.02 to 0.20 w/w)) was dissolved in purified water (0.77 to 7.7 kg, 1.00 to 10.0 w/w) and slowly added to reaction mass through charging vessel at 25±5° C.
112) The reaction mass was stirred for NLT 5 min at 25±5° C.
113) The mass was allowed to settle for NLT 5 min at 25±5° C.
114) The bottom aqueous layer was separated in clean HDPE container, and the organic layer was kept in the reactor.
115) Purified water (7.7 kg, 10.0 w/w) was charged into the above organic layer at below 30° C.
116) The reaction mass was stirred for NLT 5 min at 25±5° C.
117) The mass was allowed to settle for NLT 5 min at 25±5° C.
118) The bottom aqueous layer was separated in clean HDPE container, and the organic layer was kept in the reactor.
119) Purified water (7.7 kg, 10.0 w/w) was charged into the above organic layer at below 30° C.
120) The reaction mass was stirred for NLT 5 min at 25±5° C.
121) The mass was allowed to settle for NLT 5 min at 25±5° C.
122) The bottom aqueous layer was separated in clean HDPE container, and the organic layer was collected into fresh HDPE container. pH of the aqueous layer was adjusted to 5-7.
123) The organic layer (step no. 122) was charged back into clean and dry reactor and concentrated completely under vacuum at below 55° C.
124) The mass was cooled to 30±5° C., and the material was unloaded.
The sample was sent to QC to check the content of (2a) by GC. Acceptance criteria: Content of (2a) (By GC): NMT 35.0%.
125) The above crude (step no. 124) and Methyl tert-butyl ether (1.89 to 3.43 kg, 2.45 to 4.45 w/w) were charged into a clean dry reactor at below 30° C.
126) n-Heptane (2.63 kg, 3.42 w/w) was charged into reaction mass at below 30° C.
127) Tert-butyl amine (0.18 kg, 0.24 w/w) was slowly added into the reactor mass through GAV at 25±5° C.
128) The reaction mass was stirred for NLT 30 minutes at 25±5° C.
129) n-Heptane (6.28 kg, 8.16 w/w) was slowly added into reaction mass at below 30° C.
130) The reaction mass was stirred for NLT 60 minutes at 25±5° C.
131) The mass was filtered through a Nutsche filter, and the filtrate was collected in HDPE container.
132) The reactor was rinsed with Methyl tert-butyl ether (1.83 kg, 2.38 w/w): n-Heptane (1.83 kg, 2.38 w/w) mixture, and the wet cake was washed, and the filtrate was collected in HDPE container.
133) The Material was unloaded into double LDPE bags and placed in the HDPE container.
134) The wet material was loaded into vacuum tray drier and dried at 25±5° C. under vacuum NLT −300 mmHg until the water content comply acceptance criteria.
The sample was sent to QC for water content from 8^{th} hours onwards at every 4 hours interval until the water content comply to the acceptance criteria. Acceptance criteria: Water content NMT 5.0% w/w.
135) The Material was unloaded into double LDPE bags and placed in HDPE container. The sample was sent to QC to check the content of (2b) by HPLC and GC.
Acceptance criteria: Description Visual: Dark brown to almost white.
RSM-002-ITM09A (RSM-002 TBA salt) content by HPLC: NLT 70.0 A %,
RSM-002-ITM09A (RSM-002 TBA salt) content by GC: NLT 80.0 A %

| | | |
|---|---|---|
| Yield: | ~0.5 | kg |
| Theoretical yield: | 1.19 | kg |

| | |
|---|---|
| % Yield: | ~42.01% |

### f. (2) Preparation

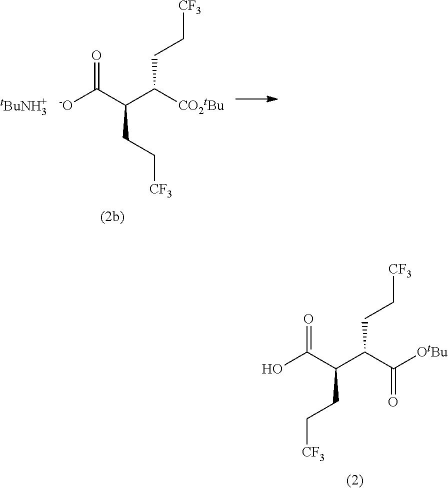

1) Methyl Tert-Butyl Ether (7.40 kg, 7.40 w/w) charged into a clean and dry reaction at 30° C.
2) (2b) (1.0 kg, 1.0 w/w) was charged into above reactor at below 30° C.
3) The reaction mass was stirred for NLT 5 minutes at below 30° C.
4) 0.5N HCl solution (Cone HCl (0.42 to 0.52 kg, 0.42 to 0.52 w/w) mixed in purified water (8.0 to 10.0 kg, 8.0 to 10.0 w/w) was slowly added into the reaction mass at 25±5° C.
5) The reaction mass was stirred for NLT 15 min at 25±5° C.
6) The reaction mass was allowed to settle for NLT 15 min at 25±5° C.
7) The bottom aqueous layer was separated and collected into HDPE drum. The top organic layer was kept inside the reactor.
8) Purified water (5.0 kg, 5.0 w/w) was charged into the above organic layer at 25±5° C.
9) The reaction mass was stirred for NLT 15 min at 25±5° C.
10) The reaction mass was allowed to settle for NLT 15 min at 25±5° C.
11) The bottom aqueous layer was separated and collected into HDPE drum. The top organic layer was kept inside the reactor.
12) Purified water (5.0 kg, 5.0 w/w) was charged into the above organic layer at 25±5° C.
13) The reaction mass was stirred for NLT 15 min at 25±5° C.
14) The reaction mass was allowed to settle for NLT 15 min at 25±5° C.
15) The bottom aqueous layer was separated and collected into HDPE drum, and the top organic layer was collected into fresh HDPE container.
16) The organic layer (step no. 15) was charged back into a clean and dry reactor.
17) The reaction mass was concentrated completely under vacuum at below 55° C.
18) The reaction mass was cooled to 30±5° C.
19) Methyl Tert-Butyl Ether (1.11 kg, 1.11 w/w) was charged into reaction mass and unloaded into HDPE container.
20) The above reaction mass was charged into clean and dry reactor at below 30° C.
21) Methyl Tert-Butyl Ether (4.81 kg, 4.81 w/w) was charged into reaction mass at below 30° C. under a nitrogen atmosphere.
22) Tertiary butylamine solution (Tertiary butylamine (0.185 kg, 0.185 w/w)) dissolved in Methyl Tert-Butyl Ether (1.48 kg, 1.48 w/w) was slowly added into the reaction mass at 25±5° C. under a nitrogen atmosphere.
23) The reaction mass was stirred at 25±5° C. for NLT 10 min.
24) n-Heptane (6.84 kg, 6.84 w/w) was slowly added into the reaction mass at 25±5° C. under a nitrogen atmosphere.
25) The reaction mass was stirred for NLT 10 minutes at 25±5° C.
26) The reaction mass was cooled to 5±5° C.
27) The reaction mass was stirred for NLT 60 minutes at 5±5° C.
28) The reaction mass was filtered through a Nutsche filter, and the filtrate was collected in HDPE container.
29) The above wet material was washed with Methyl tert butyl ether (1.48 kg, 1.48 w/w): n-heptane (1.36 kg, 1.36 w/w) mixture.
30) The material was vacuum dried for NLT 60 minutes under vacuum, and the material was unloaded into fresh HDPE container.
31) The sample was sent to QC to check content of (2a: R,R isomer) by GC and purity of (2b) (By HPLC). Acceptance criteria: (2a: R,R isomer): NMT 1.0% area (Isomeric purity (By GC)), (2b) (By HPLC): NLT 95.0% area.
32) Methyl Tert-Butyl Ether (7.40 kg, 7.40 w/w) was charged into a clean and dry reactor at below 30° C.
33) The wet material (step no. 30) was charged into above reactor at below 30° C.
34) The reaction mass was stirred for NLT 5 minutes at below 30° C.
35) 0.5N HCl solution (Conc. HCl (0.42 to 0.52 kg, 0.42 to 0.52 w/w)) mixed in purified water (8.0 to 10.0 kg, 8.0 to 10.0 w/w) was slowly added into the mass at 25±5° C.
36) The reaction mass was stirred for NLT 15 minutes at 25±5° C.
37) The reaction mass was allowed to settle for NLT 15 minutes.
38) The bottom aqueous layer was separated and collected into HDPE drum. The top organic layer was kept inside the reactor.
39) Purified water (5.0 kg, 5.0 w/w) was charged into the mass at 25±5° C.
40) The reaction mass was stirred for NLT 15 minutes at 25±5° C.
41) The reaction mass was allowed to settle for NLT 15 minutes.
42) The bottom aqueous layer was separated and collected into HDPE drum. The top organic layer was kept inside the reactor.
43) Purified water (5.0 kg, 5.0 w/w) was charged into the mass at 25±5° C.
44) The reaction mass was stirred for NLT 15 minutes at 25±5° C.
45) The reaction mass was allowed to settle for NLT 15 minutes.
46) The bottom aqueous layer was separated and collected into HDPE drum. The top organic layer was collected into fresh HDPE container.
47) The organic layer (step no. 46) was charged back into a clean and reactor at 25±5° C.
48) Activated charcoal solution (activated charcoal (0.10 kg, 0.10 w/w) mixed in Methyl tert butyl ether (0.7 kg, 0.7 w/w) was charged into above organic layer at 25±5° C.
49) The reaction mass temperature was raised to 45±5° C.
50) The reaction mass was stirred for NLT 120 min at 45±5° C.
51) The reaction mass was cooled to 25±5° C.
52) The reaction mass was cooled for NLT 60 min at 45±5° C.
53) A bed in a Nutsche filter was prepared using Celite (2.00-6.00 kg) and Methyl tert butyl ether (10.00-60.00 kg).
54) The mass was filtered through the Nutsche filter, and the filtrate was collected in fresh HDPE container.
55) The reactor was rinsed with Methyl tert-butyl ether (3.7 to 5.0 kg, 3.7 to 5.0 w/w).
56) The bed was washed with above rinsed Methyl tert-butyl ether.
57) The washed bed was vacuum dried for NLT 10 minutes, and the filtrate was collected into fresh HDPE container.
58) The above filtrate (step no. 57) was charged into the reactor through a charging line connected with Cartridge filter.
59) The reaction mass was concentrated completely under vacuum at below 55° C.
60) n-Heptane (3.42 kg, 3.42 w/w, filtered through cartridge) was charged into reaction mass at below 55° C.
61) The reaction mass was concentrated completely under vacuum at below 55° C.
62) n-Heptane (3.42 kg, 3.42 w/w, filtered through cartridge) was charged into reaction mass at below 55° C.
63) The reaction mass was concentrated completely under vacuum at below 55° C.
64) The reaction mass was cooled to 25±5° C.
65) n-Heptane (6.84 kg, 6.84 w/w, Filter through cartridge) was charged into reaction mass at below 25±5° C.
66) The reaction mass temperature was slowly raised to 50±5° C.
67) The reaction mass was stirred for NLT 10 minutes at 50±5° C. until the solution was clear.
68) The reaction mass temperature was slowly cooled to 10±5° C.
69) The reaction mass was stirred for NLT 60 min at 10±5° C.
70) The mass was filtered through the above Nutsche filter, and the filtrate was collected in fresh HDPE container.
71) The reactor was rinsed with n-Heptane (1.37 to 3.7 kg, 1.37 to 3.7 w/w, filtered through cartridge).
72) The bed was washed with above rinsed n-Heptane.
73) Compound (2) was vacuum dried NLT 60 min and then it was unloaded.
74) The sample was sent to QC to check the purity of (2) (By HPLC) and Isomeric purity (By GC).
Acceptance criteria: Purity of (2): NLT 99.0% area (By HPLC), (I-1) content: NMT 0.40% area, Impurity at RRT˜0.98: NMT 0.25% area,
   Individual unknown impurity: NMT 0.10%.
   Isomeric purity by GC: (2a: S,R isomer): NMT 0.25% w/w, (2a: R,R Isomer): NMT 0.45% w/w.

| | | |
|---|---|---|
| Yield: | ~0.5 | kg |
| Theoretical yield: | 0.83 | Kg |

| | |
|---|---|
| % Yield: | ~60.0% |

### Example 6

### Synthesis of Compound (3b) from Compounds (3-2) and (2)

Compound (3b-1): Compound (3-2) was prepared as detailed in PCT publication number WO 2021/163676, which is incorporated herein by reference. Compound (3-2) (Assay 96.5%, 1.0 eq., 459.05 g) was suspended in DMF (2.75 vol., 819.97 g). The feed container was rinsed with DMF (0.50 vol., 149.48 g), and the rinse solution was transferred to a 10 L reactor. Compound (2) (1.00 eq., 314.99 g; prepared according to Example 5) was suspended in DMF (1.25 vol., 372.25 g). The feed container was rinsed with DMF (0.50 vol., 151.67 g), and the rinse solution was transferred to the reactor. TBTU (1.30 eq., 359.20 g) was transferred as a solid to the reactor. DMF (0.50 vol., 148.90 g) was used to rinse the walls of the reactor.

The reaction mixture was cooled to 15.95° C. within 12 minutes. Triethylamine (2.55 eq., 222.03 g) was dosed to the reactor during 34 minutes while maintaining the temperature of the reaction between 15.36 and 18.43° C. The addition funnel was rinsed with DMF (0.35 vol., 104.48 g), and the rinse solution was transferred to the reactor.

The temperature of the reaction mixture was set to 19.04° C. during 8 minutes and then stirred at 20° C. After 63 minutes, IPC 1 (“In Process Control 1”, i.e. in process control performed the first time in this process) showed reaction completion. Dichloromethane (7.00 vol., 2926.67 g) was added to the reaction mixture. The batch was quenched with water and the layers separated. The rich dichloromethane layer was washed twice with water ((7.00 vol., 2205.01 g)×2) and used directly for the next step.

Dichloromethane (2.65 vol., 1109.20 g) was added to the reactor. The batch temperature was lowered to 11.84° C. and TFA (5.20 vol., 2441.51 g) was dosed during 20 minutes at 11.53-16.31° C. The reaction mixture was heated to 21.43° C. during 21 minutes and then stirred at 23° C. IPC 2 showed reaction completion after 4 hours.

The reaction mixture was concentrated at a jacket temperature of 24.72-28.09° C. until a vacuum of 180 mbar was achieved and no more distillation was observed. MTBE (7.00 vol., 1631.95 g) was dosed into the reactor during 26 minutes. 10% w/w di-potassium hydrogen-phosphate solution (17.60 w/w, 5544.37 g) was added to the reactor. The biphasic mixture was stirred, settled, and phases were separated. pH 0.94 was measured in the aqueous phase, which was then discarded. Additional 10% w/w di-potassium hydrogen-phosphate solution (17.60 w/w, 5543.73 g) was added to the reactor. The biphasic mixture was stirred, settled, and phases were separated. pH 1.54 was measured in the aqueous phase, which was then discarded. Additional 10% w/w di-potassium hydrogen-phosphate solution (17.60 w/w, 5546.54 g) was added to the reactor. The biphasic mixture was stirred, settled, and phases were separated. pH 5.70 was measured in the aqueous phase, which was then discarded.

Phosphoric acid (0.50 vol., 268.12 g) was dissolved into water (3.30 vol., 1040.91 g). Approx. 0.64 L of the phosphoric acid solution was dosed to a portion of 10% w/w di-potassium hydrogen phosphate solution (8.50 vol., 2678.30 g) until pH 5.53 was reached. A portion of this buffered solution (8.50 vol., 2679.25 g) was transferred to the reactor. The biphasic mixture was stirred, settled, and phases were separated. pH 5.62 was measured in the aqueous phase, which was then discarded.

Approx. 0.63 L of the phosphoric acid solution was dosed to a portion of 10% w/w di-potassium hydrogen phosphate solution (8.50 vol., 2679.30 g) until pH 5.55 was reached. A portion of this buffered solution (8.50 vol., 2680.40 g) was transferred to the reactor, and the leftover was discarded. The biphasic mixture was stirred, settled, and phases were separated. pH 5.57 was measured in the aqueous phase, which was then discarded.

IPC 3 was performed and a residual TFA level of 0.02 mg/mL was found in the organic phase.

Water (8.00 vol., 2520.85 g) was added to the reactor. The biphasic mixture was stirred, settled, and phases were separated. pH 5.36 was measured in the aqueous phase, which was then discarded.

The organic phase was filtered through a 10 μm filter to the reactor at 20° C., and the bottle was rinsed with MTBE (0.80 vol., 186.26 g).

A DABCO solution was prepared by dissolving DABCO (2.25 eq., 217.46 g) in isopropyl acetate (6.90 vol., 1892.84 g) and MTBE (3.50 vol., 816.52 g). The DABCO solution was dosed into the reactor during 37 minutes. The bottle was rinsed with MTBE (0.4 vol., 93.94 g). The suspension was stirred during 121 minutes at 19.89-20.00° C. and then filtered on a 3 L porosity 3 sintered glass filter. The filter cake was rinsed twice with MTBE (2×4.40 vol., 1028.54 g & 1026.48 g) and then dried at 25° C. during 5.5 days. A yield of 101% (597.6 g) was measured. Compound (3b-1) was obtained with an HPLC Purity of 98.9% a/a.

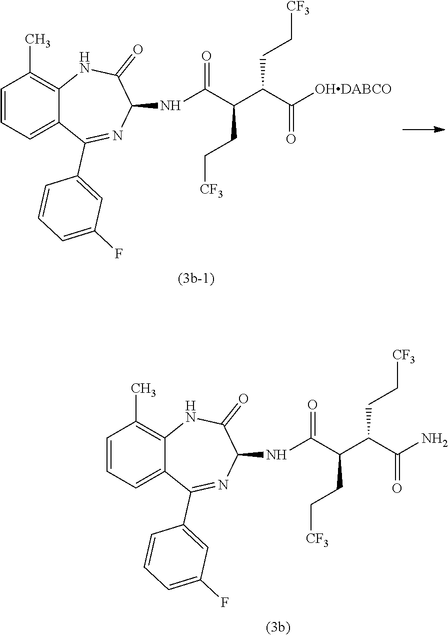

Compound (3b): compound (3b-1) (1.0 eq., 588.0 g) was transferred to the reactor as a suspension in DMF (2.80 vol., 1555.70 g) and transferred to a 30 L reactor. The feed container was rinsed with 2 portions of DMF (2.00 vol. & 1.10 vol., 1114.61 g & 611.2 g), and the rinse solutions were transferred to the reactor.

HOBt (2.50 eq., 327.1 g) was suspended in DMF (1.20 vol., 667.5 g) and transferred to the reactor. The feed container was rinsed with DMF (1.10 vol., 610.4 g), and the rinse solution was transferred to the reactor.

The temperature of the reaction mixture was set during 14 minutes until 12.1° C. was reached and then a 2.0 M solution of ammonia in isopropanol (3.30 vol., 1523.1 g) was transferred to the reactor.

EDC·HCl (2.50 eq., 410.1 g) was suspended in DMF (3.50 vol., 1954.2 g) and transferred to the reactor. The feed container was rinsed with DMF (3.15 vol., 1750.6 g), and the rinse solution was transferred to the reactor.

The reaction mixture was stirred between 11.9 and 12.2° C. during 43 minutes, heated to 19.2° C. during 43 minutes and then stirred between 19.4 and 20.3° C. After 21 h23 minutes, IPC 1 (“In Process Control 1”, i.e. in process control performed the first time in this process) showed reaction completion.

Water (30.00 vol., 17638.1 g) was added during 89 minutes while maintaining the temperature between 16.3 and 21.2° C. The suspension was filtered on a 3 L P3 sintered glass filter. The filter cake was rinsed twice with water (2×5.00 vol., 2940.3 g & 2940.3 g) and then dried at 50° C. during 5 days. A yield of 88% (430.7 g) was determined on the filter. IPC 2 was conducted for information and showed a HPLC purity of 98.9% a/a.

Crude (3b) was dissolved in acetic acid (4.00 vol., 1772.16 g) and transferred to a 10 L reactor. The feed container was rinsed with acetic acid (1.00 vol., 443.11 g), and the rinse solution was transferred to the reactor. The temperature of the reaction mixture was set to 19.36° C.

Water (5.50 vol., 2322.44 g) was transferred to the reactor during 25 minutes while maintaining the temperature between 14.92 and 16.65° C. The resulting suspension was stirred between 14.67 and 16.40° C. during 15 minutes and then filtered on a 3 L P3 sintered glass filter. The filter cake was washed twice with water (2×5.00 vol., 2110.18 g & 2112.32 g) and then dried under vacuum at 50° C. during 4 days. A yield of 82% (404.3 g) was determined on the filter. IPC 3 was conducted and showed an HPLC purity of 99.80% a/a, and a residual acetic acid level of 0.10% w/w.

### Example 7

### Synthesis of Compound (3a) from Compounds (3-1) and (2)

Compound (3-1) was prepared as detailed in Reider, P. J. et al., J. Org. Chem., 52:955-957 (1987), which is incorporated herein by reference.

Compound (3a) was prepared from compounds (3-1) and (2) as detailed in PCT publication number WO 2012/129353, which is incorporated herein by reference.

What is claimed is:

1. A salt represented by the structure of Formula (I): 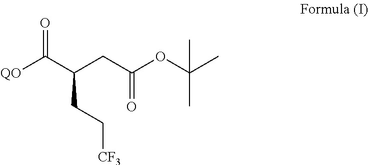
wherein Q is an ammonium cation.

2. The salt of claim 1, wherein said ammonium cation is a primary, secondary or tertiary ammonium cation.

3. The salt of claim 2, wherein the primary ammonium cation comprises a cycloalkyl moiety.

4. The salt of claim 2 or 3, wherein the primary ammonium cation is cyclohexyl, cyclopentyl, cycloheptyl or cyclooctyl ammonium cation.

5. The salt of claim 4, wherein the primary ammonium cation is cyclohexyl ammonium cation.

6. The salt of claim 2, wherein the secondary ammonium cation is a di-cyclohexyl, di-cyclopentyl, di-cycloheptyl or di-cyclooctyl ammonium cation.

7. The salt of claim 2, wherein the tertiary ammonium cation is a di-isopropyl ethyl, diethyl isopropyl or N-methylmorpholine ammonium cation.

8. The salt according to any one of the preceding claims, wherein the salt is stable for 6-12 months.

9. The salt of claim 8, wherein the salt is stable for 6 months.

10. A method of preparing compound (3a) or (3b), comprising:
Step A: 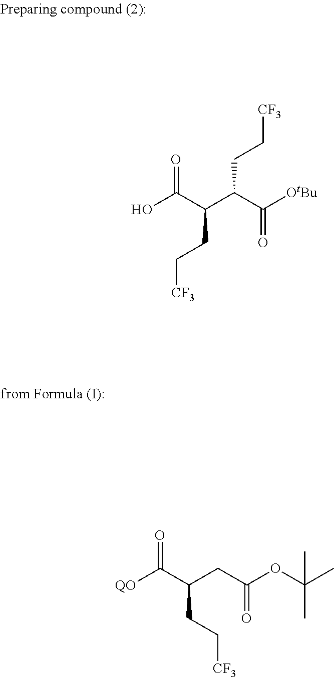
wherein
Q is an ammonium cation;
and
Step B:

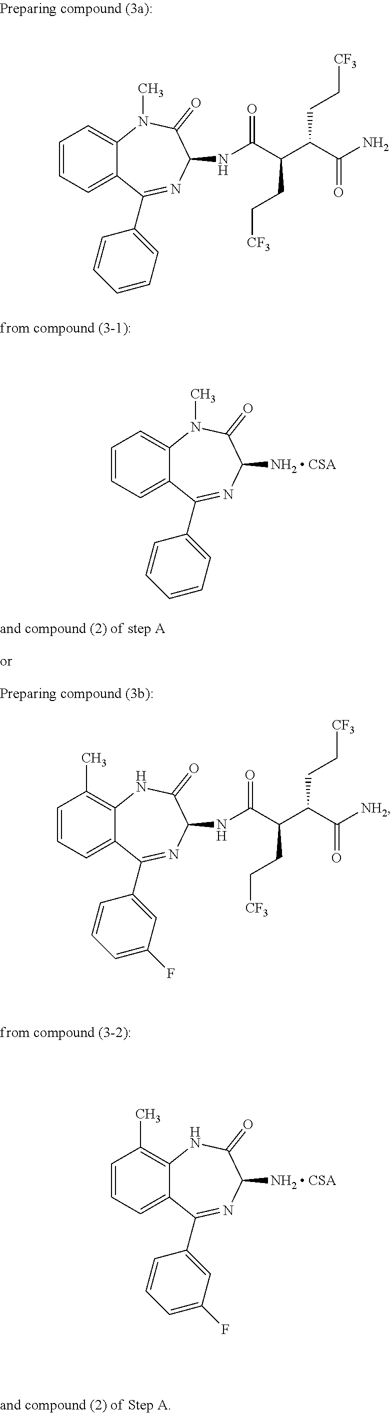

11. A method of preparing compound (2) from Formula (I), comprising: 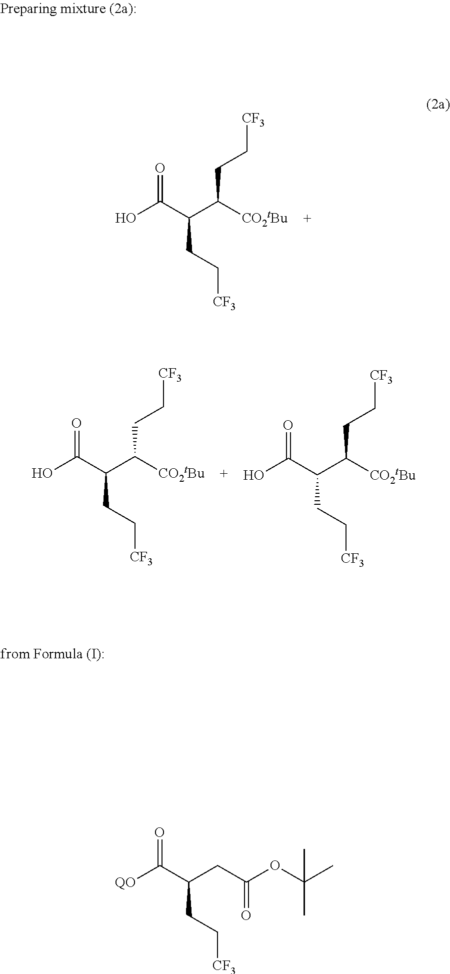
wherein
Q is an ammonium cation; and

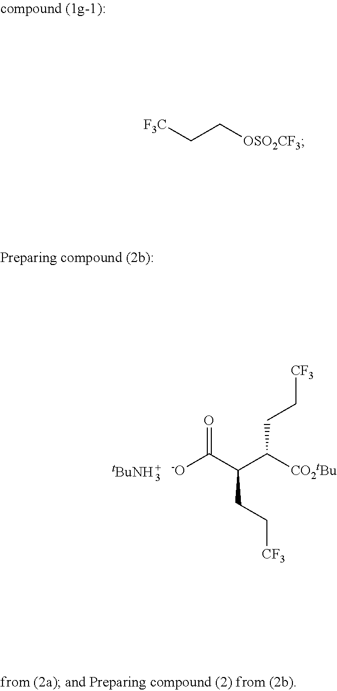

12. The method of claim 10 or 11, wherein Formula (I) is prepared via the following method, comprising:

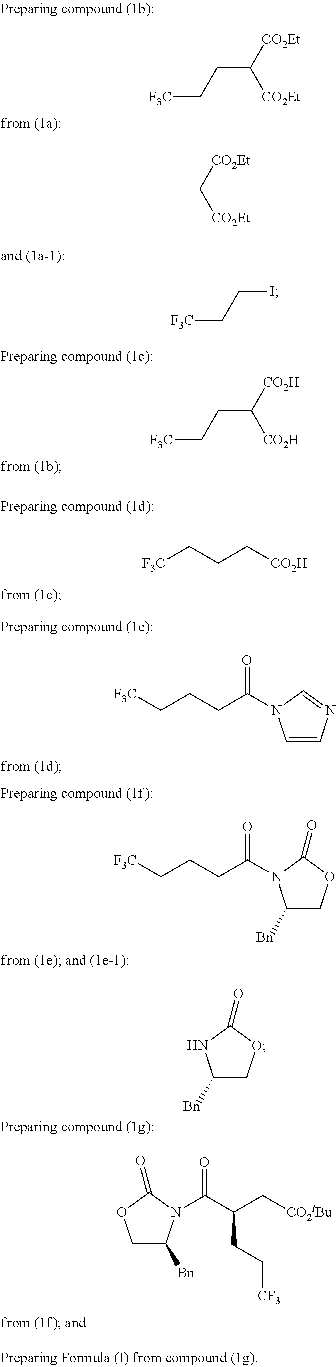

13. The method of claim 12, wherein the step of preparing Formula (I) from compound (1g) comprises:

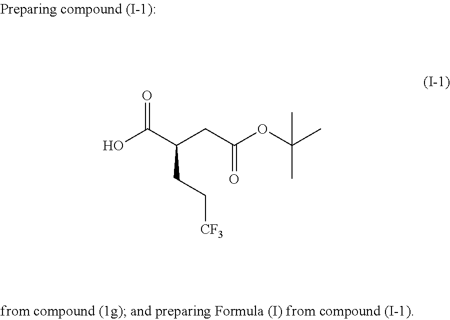 [SMILES: CC(C)(C)OC(=O)C[C@@H](CCC(F)(F)F)C(=O)O]

14. The method according to any one of claims 10-13, wherein Q of Formula (I) is a cyclohexyl ammonium cation.
